(19) United States
(12) Patent Application Publication
(10) Pub. No.: US 2022/0365069 A1
(43) Pub. Date: Nov. 17, 2022

(54) ATP-BASED CELL SORTING AND HYPERPROLIFERATIVE CANCER STEM CELLS

(71) Applicant: LUNELLA BIOTECH, INC., Ottawa, Ontario (CA)

(72) Inventors: Michael P. LISANTI, Didsbury Village, Greater Manchester (GB); Federica SOTGIA, Didsbury Village, Greater Manchester (GB); Marco FIORILLO, Manchester, Greater Manchester (GB)

(21) Appl. No.: 17/642,935

(22) PCT Filed: Sep. 14, 2020
(86) PCT No.: PCT/IB2020/058524

§ 371 Date: Mar. 14, 2022

Related U.S. Application Data
(60) Provisional application No. 62/900,139, filed on Sep. 13, 2019.

(51) Int. Cl. G01N 33/50; G01N 33/574; G01N 33/573

(52) U.S. Cl. CPC G01N 33/5011; G01N 33/57484; G01N 33/5735

(57) ABSTRACT

High mitochondrial ATP is a metabolic trait that confers hyper-proliferation, sternness, anchorage-independence, anti-oxidant capacity and multi-drug resistance in cancer cells. Under the present approach, intracellular ATP levels may be used as a metabolic biomarker to identify, separate, and purify an aggressive and hyper-proliferative cancer stem cell (“CSC”) phenotype. Further, ATP may be combined with other CSC markers, e.g., CD44 or ALDH-activity, to beneficially fractionate the CSC population into sub-populations. For example, ATP-high/ CD44-high CSC sub-populations showed twice the level of anchorage-independent growth compared to ATP-low/CD44-high CSC sub-populations. Also disclosed are complementary bioinformatic data that implicate mitochondrial ATP synthesis in stemness, metastasis, and the detection of circulating tumor cells (“CTCs”), and a five-member, ATP-related metastasis gene-signature (ABCA2, ATP5F1C, COX20, NDUFA2 and UQCRB). The gene signature of the present approach may be used to identify CSCs having a dramatic increase in cell migration and invasion in vitro capacity, as well as spontaneous metastasis in vivo. The present approach also provides a cellular platform for systematically targeting sternness, multi-drug resistance, and metastasis in cancer cells.

### RELATED APPLICATIONS

This application claims the benefit of U.S. provisional patent application 62/900,139, filed Sep. 13, 2019, and incorporated herein by reference in its entirety.

### FIELD

The present disclosure relates to ATP-based cell sorting to identify, separate, and treat metabolically-hyperactive, aggressive, and hyper-proliferative cancer stem cell (“CSC”) phenotypes, and for preventing or reducing the likelihood of metastasis.

### BACKGROUND

Researchers have struggled to develop new anti-cancer treatments. Conventional cancer therapies (e.g. irradiation, alkylating agents such as cyclophosphamide, and anti-metabolites such as 5-Fluorouracil) have attempted to selectively detect and eradicate fast-growing cancer cells by interfering with cellular mechanisms involved in cell growth and DNA replication. Other cancer therapies have used immunotherapies that selectively bind mutant tumor antigens on fast-growing cancer cells (e.g., monoclonal antibodies). Unfortunately, tumors often recur following these therapies at the same or different site(s), indicating that not all cancer cells have been eradicated. Relapse may be due to insufficient chemotherapeutic dosage and/or emergence of cancer clones resistant to therapy. Hence, novel cancer treatment strategies are needed.

Advances in mutational analysis have allowed in-depth study of the genetic mutations that occur during cancer development. Despite having knowledge of the genomic landscape, modern oncology has had difficulty with identifying primary driver mutations across cancer subtypes. The harsh reality appears to be that each patient's tumor is unique, and a single tumor may contain multiple divergent clone cells. What is needed, then, is a new approach that emphasizes commonalities between different cancer types. Targeting the metabolic differences between tumor and normal cells holds promise as a novel cancer treatment strategy. An analysis of transcriptional profiling data from human breast cancer samples revealed more than 95 elevated mRNA transcripts associated with mitochondrial biogenesis and/or mitochondrial translation. Additionally, more than 35 of the 95 upregulated mRNAs encode mitochondrial ribosomal proteins (MRPs). Proteomic analysis of human breast cancer stem cells likewise revealed the significant overexpression of several mitoribosomal proteins as well as other proteins associated with mitochondrial biogenesis.

Mitochondria are extremely dynamic organelles in constant division, elongation and connection to each other to form tubular networks or fragmented granules in order to satisfy the requirements of the cell and adapt to the cellular microenvironment. The balance of mitochondrial fusion and fission dictates the morphology, abundance, function and spatial distribution of mitochondria, therefore influencing a plethora of mitochondrial-dependent vital biological processes such as ATP production, mitophagy, apoptosis, and calcium homeostasis. In turn, mitochondrial dynamics can be regulated by mitochondrial metabolism, respiration and oxidative stress. Thus, it is not surprising that an imbalance of fission and fusion activities has a negative impact on several pathological conditions, including cancer. Cancer cells often exhibit fragmented mitochondria, and enhanced fission or reduced fusion is often associated with cancer, although a comprehensive mechanistic understanding on how mitochondrial dynamics affects tumorigenesis is still needed.

An intact and enhanced metabolic function is necessary to support the elevated bioenergetic and biosynthetic demands of cancer cells, particularly as they move toward tumor growth and metastatic dissemination. Not surprisingly, mitochondria-dependent metabolic pathways provide an essential biochemical platform for cancer cells, by extracting energy from several fuels sources.

Cancer stem-like cells are a relatively small sub-population of tumor cells that share characteristic features with normal adult stem cells and embryonic stem cells. As such, CSCs are thought to be a ‘primary biological cause’ for tumor regeneration and systemic organismal spread, resulting in the clinical features of tumor recurrence and distant metastasis, ultimately driving treatment failure and premature death in cancer patients undergoing chemo- and radio-therapy. Evidence indicates that CSCs also function in tumor initiation, as isolated CSCs experimentally behave as tumor-initiating cells (TICs) in pre-clinical animal models. As approximately 90% of all cancer patients die pre-maturely from metastatic disease world-wide, there is a great urgency and unmet clinical need, to develop novel therapies for effectively targeting and eradicating CSCs. Most conventional therapies do not target CSCs and often increase the frequency of CSCs, in the primary tumor and at distant sites.

Recently, energetic metabolism and mitochondrial function have been linked to certain dynamics involved in the maintenance and propagation of CSCs, which are a distinguished cell sub-population within the tumor mass involved in tumor initiation, metastatic spread and resistance to anti-cancer therapies. For instance, CSCs show a peculiar and unique increase in mitochondrial mass, as well as enhanced mitochondrial biogenesis and higher activation of mitochondrial protein translation. These behaviors suggest a strict reliance on mitochondrial function. Consistent with these observations, an elevated mitochondrial metabolic function and OXPHOS have been detected in CSCs across multiple tumor types.

One emerging strategy for eliminating CSCs exploits cellular metabolism. CSCs are among the most energetic cancer cells. Under this approach, a metabolic inhibitor is used to induce ATP depletion and starve CSCs to death. So far, the inventors have identified numerous FDA-approved drugs with off-target mitochondrial side effects that have anti-CSC properties and induce ATP depletion, including, for example, the antibiotic Doxycycline, which functions as a mitochondrial protein translation inhibitor. Doxycycline, a long-acting Tetracycline analogue, is currently used for treating diverse forms of infections, such as acne, acne rosacea, and malaria prevention, among others. In a recent Phase II clinical study, pre-operative oral Doxycycline (200 mg/day for 14 days) reduced the CSC burden in early breast cancer patients between 17.65% and 66.67%, with a near 90% positive response rate.

However, certain limitations restrain the use of sole anti-mitochondria agents in cancer therapy, as adaptive mechanisms can be adopted in the tumor mass to overcome the lack of mitochondrial function. These adaptive mechanisms include, for example, the ability of CSCs to shift from oxidative metabolism to alternate energetic pathways, in a multi-directional process of metabolic plasticity driven by both intrinsic and extrinsic factors within the tumor cells, as well as in the surrounding niche. Notably, in CSCs the manipulation of such metabolic flexibility can turn as advantageous in a therapeutic perspective. What is needed, then are therapeutic approaches that either prevent these metabolic shifts, or otherwise take advantage of the shift to inhibit cancer cell proliferation.

Adenosine-5′-triphosphate (ATP) is the bio-energetic “currency” of all living cells and organisms. Chemically, ATP is a nucleoside triphosphate, which contains adenine, a ribose sugar, and three phosphate groups. ATP cleavage at its terminal phosphate group, produces two main reaction products, ADP and inorganic phosphate (Pi), thereby releasing high levels of stored energy. In eukaryotic cells, mitochondria generate the vast amount of ATP via the TCA cycle and oxidative phosphorylation (OXPHOS), while glycolysis contributes a minor amount of ATP. Mitochondrial dysfunction induces ATP-depletion, resulting in mitochondrial-driven apoptosis (cell death).

In MCF7 breast cancer cells, mitochondrial-driven OXPHOS contributes to 80% of ATP production, while glycolysis contributes the remaining 20%. Therefore, like normal cells, cancer cells are still highly dependent on mitochondrial ATP production. However, it remains largely unknown how ATP levels in cancer cells contribute to “stemness” and cell cycle progression, as well as their ability to undergo anchorage-independent growth, a characteristic feature of metastatic spread.

Because of the central importance of ATP as a barometer of cell metabolism, many luminescent and fluorescent probes have been developed to measure and track ATP levels, in response to various cellular stimuli. For example, ATP-Red 1 (CAS#: 1847485-97-5, IUPAC Name: [2-[3′, 6′-bis(diethylamino)-3-oxospiro[isoindole-1,9′-xanthene]-2-yl]phenyl]boronic acid) is a vital dye that is only fluorescent when bound to ATP, and does not recognize ADP or other nutrients. ATP-Red 1 allows for the dynamic visualization of ATP levels in living cells and tissues.

An object of this disclosure is to describe a viable ATP-depletion strategy for targeting and eradicating even the “fittest” cancer cells.

It is another object of this disclosure to describe unique compositions of cells of a particular, hyper-proliferative phenotype.

It is another object of this disclosure to identify new anti-cancer therapeutic approaches involving new pharmaceutical compounds that metabolically starve CSCs by targeting mitochondria and driving ATP depletion.

### SUMMARY

The present approach describes the use of a fluorescent ATP imaging probe to metabolically fractionate a cancer cell population, and separate a hyper-proliferative cell sub-population. The resulting composition may be used for numerous advantageous purposes, ranging from rapid drug development and screening, to predicting and preventing metastasis and drug resistance. The present approach also provides a 5-gene signature prognostic of metastasis in a cancer, and methods for metabolic fractionation of cancer cells, and diagnosis and prevention of metastasis.

Bioenergetic cell “stratification” employing an ATP-based biomarker may be used to isolate the “fittest” cancer cells, for identification, diagnosis, treatment, and therapeutic drug development. In particular, a fluorescent ATP imaging probe, such as Biotracker ATP-Red 1, may be used to stain a cell population, and the resulting ATP-based fluorescence may be used to metabolically fractionate the population into ATP-high and, if desired, bulk and ATP-low sub-populations. Using this novel approach, the data disclosed herein includes the first evidence that high levels of mitochondrial ATP are a primary determinant of aggressive cancer cell behavior(s), including spontaneous metastasis.

There is a considerable amount of phenotypic diversity and metabolic heterogeneity in the cancer cell population. This heterogeneity allows the “fittest” cancer cells to escape current treatment modalities, resulting in tumor recurrence and distant metastasis, secondary to drug resistance.

High intracellular ATP levels may be used as a metabolic biomarker for an aggressive and hyper-proliferative cancer cell phenotype. Under the present approach, a fluorescent ATP marker, such as the vital dye BioTracker™ ATP-Red 1 (EMD Millipore Corporation, Burlington, Massachusetts), may be used to quantify mitochondrial ATP levels in a cancer cell population, and isolate ATP-high and ATP-low cancer cell sub-populations by flow cytometry. Phenotypic analysis of these sub-populations shows that high mitochondrial ATP is a metabolic trait that confers hyper-proliferation, sternness, anchorage-independence, anti-oxidant capacity, and multi-drug resistance in cancer cells. Quantitatively similar results were obtained with four human breast cancer cell lines, MCF7, T47D, MDA-MB-231 and MDA-MB-468.

By combining ATP with other CSC markers, e.g., CD44 or ALDH-activity, the CSC population may be advantageously fractionated into two sub-populations. The CD44-high/ATP-high sub-populations have about twice the level of anchorage-independent growth compared to CD44-high/ATP-low sub-populations. Thus, CD44-high/ATP-low cancer cells represent a more dormant CSC population. Importantly, these results indicate that ATP levels may be a functional regulator of dormancy in CSCs.

The present approach also includes complementary bioinformatic data that implicate mitochondrial ATP synthesis in stemness, metastasis, and the detection of circulating tumor cells (CTCs). Disclosed herein is a five-member, ATP-related metastasis gene-signature comprising ABCA2, ATP5F1C, COX20, NDUFA2 and UQCRB. In accordance with these metastasis-based clinical findings, ATP-high MDA-MB-231 cells showed dramatic increases in their capacity to undergo both cell migration and invasion in vitro, as well as spontaneous metastasis in vivo.

Thus, the present approach provides a new cellular platform for systematically identifying, studying, and targeting stemness, multi-drug resistance, and metastasis in cancer cells. This disclosure also mechanistically explains the positive therapeutic benefits of i) nutrient fasting and ii) caloric-restriction mimetics, for improving cancer therapy, by inducing ATP-depletion.

In embodiments of the present approach, vital dye ATP-Red 1 is used as a molecular probe to identify and isolate ATP-high and ATP-low sub-populations of cells, and more specifically, cancer cells and CSCs. The ATP-high sub-population of cancer cells are larger, more energetic, hyper-proliferative and undergo anchorage-independent growth, consistent with a more “stem-like” phenotype. These ATP-rich cells may be targeted with ATP-depletion therapy, to eradicate the energetically “fittest” CSCs, reduce drug resistance, and prevent metastasis.

Some embodiments of the present approach may take the form of a purified composition of hyper-proliferative cancer stem cells, in the form of a sub-population of cells from a human cancer cell population, the cancer cell population expressing a range of fluorescent signals in response to a fluorescent adenosine triphosphate (ATP) imaging probe, and the sub-population of cells expressing an upper portion of the range of ATP-based fluorescent signals. The fluorescent ATP imaging probe may be, for example, BioTracker ATP-Red 1. The upper portion, or ATP-high sub-population, may be the top 10%, 5%, or 1% of ATP-based fluorescent signals, depending on the embodiment. Other portions may be used. In some embodiments, the composition is positive for a CD44 marker. In some embodiments, the composition is positive for an ALDH marker. In some embodiments, the composition is frozen.

In some embodiments, the present approach may take the form of a purified cell composition comprising a cancer stem cell sub-population stained with a fluorescent ATP imaging probe and expressing a target portion of an ATP-based fluorescent signal range of a cancer cell population. The cancer cell population expresses a range of ATP-based fluorescent signals, and the target portion of the ATP-based fluorescent signal range may be an upper portion of the ATP-based fluorescent signals (e.g., ATP-high sub-population) and/or a lower portion of the ATP-based fluorescent signals (e.g., ATP-low sub-population). The target portion may be the top or bottom 10%, 5%, or 1% of ATP-based fluorescent signals, or other portion as selected.

Some embodiments may take the form of a purified composition of cells obtained by staining a human cancer cell population with a fluorescent ATP imaging probe, separating a fraction of the human cancer cell population having a target portion of ATP-based fluorescent signals, and purifying the separated cells. The target portion may be, for example, the top 10% of ATP-based fluorescent signals, the top 5% of ATP-based fluorescent signals, the bottom 10% of ATP-based fluorescent signals, the bottom 5% of ATP-based fluorescent signals, etc. The separated cells are positive for one of a CD44 marker and an ALDH marker.

Some embodiments may take the form of a method of ATP-based cell fractionation. Cells in a cell population may be stained with a fluorescent ATP imaging probe that fluoresces when bound to ATP. The ATP-based fluorescent signals of the stained cells in the cell population may be measured. The stained cells may be separated based on a target portion of ATP-based fluorescent signals. Fluorescence-activated cell sorting (FACS) and gating of the target portion of ATP-based fluorescent signals may be used to separate the stained cells. The gates may be set to collect the stained cells having the top 10% of measured fluorescent signals, and/or the stained cells having the bottom 10% of measured fluorescent signals. It should be appreciated that other percentages may be used. The cell population may be derived from, for example, of blood, urine, saliva, tumor tissue, non-cancerous tissue, or a metastatic lesion. Some embodiments may further include measuring ALDH activity of separated cells, measuring anchorage-independent growth of separated cells, measuring the mitochondrial mass of separated cells, measuring the glycolytic and oxidative mitochondrial metabolism of separated cells, measuring the cell cycle progression and proliferative rate of separated cells, and measuring the poly-ploidy of separated cells.

Embodiments of the present approach may take the form of a method for separating and collecting metabolically-active cells from a cell population. Cells in a cell population may be stained with an ATP-labeling dye that fluoresces when bound to ATP. The fluorescent signals of the stained cells may be measured in the cell population, and then the stained cells based on the measured fluorescent signals. At least a portion of the separated cells, having a measured fluorescent signal one of above a predetermined threshold and below a predetermined threshold, may then be collected, such as by using a FACS machine. The predetermined threshold comprises a percentage of an upper portion of the measured fluorescent signals, such as, for example, the top 25%, the top 20%, the top 15%, the top 10%, the top 5%, the top 2%, and the top 1%. Other percentages may be used without departing from the present approach. In some embodiments, the separated cells may be further separated based on a second marker, such as CD44(+), CD133(+), ESA(+), ALDEFLOUR(+), MitoTracker-High, EpCAM(+), CD90(+), CD34(+), CD29(+), CD73(+), CD90(+), CD105(+), CD106(+), CD166(+), and Stro-1(+). Other markers may be used, without departing from the present approach. The second marker may take the form of an antibody coated on magnetic beads, in some embodiments.

The present approach may also take the form of a method for identifying and treating cancer stem cells in a biologic sample. A biologic sample may be obtained from a patient, and then cells in the biologic sample may be stained with an ATP-labeling dye, wherein the ATP-labeling dye fluoresces when bound to ATP. The fluorescent signals of the stained cells in the cell population may be measured, and then compared to a predetermined threshold indicating the presence of cancer stem cells. If the measured fluorescent signals exceeds the predetermined threshold, an ATP-depletion therapeutic may be administered to the patient. The ATP-depletion therapeutic may be, for example, Doxycycline, Tigecycline, Azithromycin, Pyrvinium pamoate, Atovaquone, Bedaquiline, Niclosamide, Irinotecan, Actinonin, CAPE, Berberine, Brutieridin, Melitidin, Oligomycin, AR-C155858, a Mitoriboscin, a Mitoketoscin, a Mitoflavoscin, a TPP-derivative, dodecyl-TPP, 2-Butene-1,4-bis-TPP, or the combination of Doxycycline, Azithromycin and Ascorbic acid.

In some embodiments, the present approach may take the form of a method of testing a candidate compound for anti-cancer activity. A cancer cell population may be stained with an ATP-labeling dye that fluoresces when bound to ATP, such as BioTracker ATP-Red 1. The ATP-based fluorescent signals of the stained cells may be measured, and the stained cells may be separated based on a target portion of ATP-based fluorescent signals to prepare a hyper-active cancer cell sub-population. The candidate compound may be administered to the hyper-active cancer cell sub-population; the effect of the candidate compound on the hyper-active cancer cell sub-population may be measured. The ATP-labeling dye may be BioTracker ATP-Red 1. The target portion of ATP-based fluorescent signals may be, for example, the top 25%, the top 20%, the top 15%, the top 10%, the top 5%, the top 2%, and the top 1%. In some embodiments, the hyper-active cancer cell sub-population is positive for one of a CD44 marker an ALDH marker. Embodiments may also involve measuring ALDH activity of the hyper-active cancer cell sub-population, measuring anchorage-independent growth of the hyper-active cancer cell sub-population cells, measuring the mitochondrial mass of the hyper-active cancer cell sub-population, measuring the glycolytic and oxidative mitochondrial metabolism of the hyper-active cancer cell sub-population, measuring the cell cycle progression and proliferative rate of the hyper-active cancer cell sub-population, and measuring the poly-ploidy of the hyper-active cancer cell sub-population.

The present approach may also take the form of a method of diagnosing and preventing a risk of metastasis in a cancer patient. The expression levels of the 5-member gene signature of ABCA2, ATP5F1C, COX20, NDUFA2, and UQCRB, in a biologic sample of the patient's cancer may be determined, and then compared to baseline expression levels of ABCA2, ATP5F1C, COX20, NDUFA2, and UQCRB, in a non-cancerous biologic sample from the patient. If the detected expression levels exceed the baseline expression levels, an ATP-depletion compound may be administered to the patient. The ATP-depletion compound may be, for example, Doxycycline, Tigecycline, Azithromycin, Pyrvinium pamoate, Atovaquone, Bedaquiline, Niclosamide, Irinotecan, Actinonin, CAPE, Berberine, Brutieridin, Melitidin, Oligomycin, AR-C155858, a Mitoriboscin, a Mitoketoscin, a Mitoflavoscin, a TPP-derivative, dodecyl-TPP, 2-Butene-1,4-bis-TPP, or a combination of Doxycycline, Azithromycin and Ascorbic acid.

Some embodiments may take the form of a kit for identifying circulating tumor cells in a biologic sample. The kit may include reagents for identifying an up-regulation of ABCA2, ATP5F1C, COX20, NDUFA2, and UQCRB in the biologic sample, such as antibodies directed to the proteins encoding those genes. The kit may be used for, as an example, a liquid biopsy procedure to detect CTCs.

The present approach may also take the form of a method for detecting circulating tumor cells (CTCs) in a biologic sample. The expression levels of ABCA2, ATP5F1C, COX20, NDUFA2, and UQCRB, in the biologic sample may be determined, and then CTCs are identified as present if the determined expression levels are upregulated relative to a control. The biologic sample may be, as examples, blood, urine, saliva, tumor tissue, non-cancerous tissue, or a metastatic lesion. The sample may be further processed to separate ATP-high cells, using the methods described herein.

These and other embodiments will be apparent to the person having an ordinary level of skill in the art in view of this description, the claims appended hereto, and the applications incorporated by reference herein.

### BRIEF DESCRIPTION OF THE DRAWINGS

FIG. 1A shows a HeatMap of ATP-related genes that were transcriptionally upregulated under both 3D growth conditions (anchorage-independent and in vivo tumors), all relative to 2D-adherent growth. FIGS. 1B and 1C show volcano plots for the GSE2034 and GSE59000 GEO DataSets. FIG. 1D shows a Venn diagram intersecting the two breast cancer metastasis GEO DataSets (GSE2034 and GSE59000), used to identify ATP-related genes highly upregulated in both data sets, as prognostic biomarkers of metastasis.

FIGS. 2A-2N are data plots showing the positive correlation of APT5F1C versus the genes CDH1, ALDH2, SOX2, VIM, CD44, EPCAM, MKI67, RRP1B, CXCR4, VCAM1, CDK1, CDK2, CDK4, and CDK6, respectively. FIGS. 20-2Q are data plots showing the positive correlation of APT5F1C versus UQCRB, COX20, and NDUFA2, respectively.

FIG. 3A shows a Kaplan-Meier curve for ER(+), recurrence-free survival (N=3,082), FIG. 3B shows a Kaplan-Meier curve for ER(+), distant metastases-free survival (N=1,395), and FIG. 3C shows a Kaplan-Meier curve for ER(+), lymph node negative, Tamoxifen-treated, relapse-free survival (N=471).

FIG. 4A shows a HeatMap of an ATP-ABC gene expression profile, and FIG. 4B shows a HeatMap of the OXPHOS gene expression profile. FIG. 4C is a Western blot analysis of MDA-MB-231 cells in the ATP-high and ATP-low sub-populations.

FIG. 5A illustrates an embodiment of the metabolic fractionation procedure according to the present embodiment. FIG. 5B illustrates an example of metabolic fractionation of MCF7 cells with ATP-Red 1, to isolate ATP-high (top 5%) and ATP-low (bottom 5%) cell sub-populations.

FIGS. 6A and 6B show results from a continuous, real-time assay system on cell proliferation of three cell sub-populations (ATP-low 5%, Bulk 5%, ATP-high 5%).

FIG. 7A is a bar graph that shows changes in luminescence of cells in the ATP-high MCF7 sub-population, and FIG. 7B shows mammosphere formation assay results for ATP-high, bulk, and ATP-low sub-populations. FIG. 7C shows comparative images of the cell sub-populations after the assay. FIG. 7D shows signal strength for CD44 and ALDH positive sub-populations, and FIG. 7E shows the results of the Cell-Titer-Glo of this analysis.

FIGS. 8A and 8B show results relating to the metabolic profiling of 3D-mammospheres and ATP-high MCF7 cells.

FIGS. 9A and 9B show Cell-Titer-Glo and 3D mammosphere formation results for ATP-high and ATP-low sub-populations of MCF7, T47D, MDA-MB-231 and MDA-MB-468 cells, using a 10% gate.

FIG. 10A shows luminescence in ATP-high and ATP-low sub-populations (10%) in a MCF7 cell population after a 24-hour period. FIGS. 10B through 10E show the results of metabolic flux analysis on the ATP-high and ATP-low sub-populations.

FIGS. 11A-11D show cell cycle progression in MCF7, T47D, MDA-MB-468, and MDA-MB-231 cells, using FACS analysis with propidium iodide to detect DNA-content.

FIG. 12A shows drug resistance results for the ATP-low sub-population (bottom 5%), and FIG. 12B shows drug resistance results for the ATP-high subpopulation (top 5%).

FIGS. 13A and 13B show mammosphere assay formation results for double-labelled cells (CD44 and ATP) in MCF7 cells and MDA-MB-231 cells, respectively, and FIGS. 13C and 13D show mammosphere assay formation results for double-labelled cells (ALDH-activity and ATP) in in MCF7 cells and MDA-MB-231 cells, respectively.

FIGS. 14A and 14B show the results of a migration and invasion assay on MDA-MB-231 cells in an ATP-high sub-population.

FIG. 15 shows the results of the spontaneous metastasis in vivo CAM assay.

FIGS. 16A-16C show luminescence change, cell cycle progression, and mammosphere formation assay results of Tempo-ATP MCF7 cells, respectively.

### DESCRIPTION

The following description illustrates embodiments of the present approach in sufficient detail to enable practice of the present approach. Although the present approach is described with reference to these specific embodiments, it should be appreciated that the present approach can be embodied in different forms, and this description should not be construed as limiting any appended claims to the specific embodiments set forth herein. Rather, these embodiments are provided so that this disclosure will be thorough and complete, and will fully convey the scope of the present approach to those skilled in the art.

This description uses various terms that should be understood by those of an ordinary level of skill in the art. The following clarifications are made for the avoidance of doubt. The terms “treat,” “treated,” “treating,” and “treatment” include the diminishment or alleviation of at least one symptom associated or caused by the state, disorder or disease being treated, in particular, cancer. In certain embodiments, the treatment comprises diminishing and/or alleviating at least one symptom associated with or caused by the cancer being treated, by the compound of the invention. In some embodiments, the treatment comprises causing the death of a category of cells, such as CSCs, of a particular cancer in a host, and may be accomplished through preventing cancer cells from further propagation, and/or inhibiting CSC function through, for example, depriving such cells of mechanisms for generating energy. For example, treatment can be diminishment of one or several symptoms of a cancer, or complete eradication of a cancer. As another example, the present approach may be used to inhibit mitochondrial metabolism in the cancer, eradicate (e.g., killing at a rate higher than a rate of propagation) CSCs in the cancer, eradicate TICs in the cancer, eradicate circulating tumor cells in the cancer, inhibit propagation of the cancer, target and inhibit CSCs, target and inhibit TICs, target and inhibit circulating tumor cells, prevent (i.e., reduce the likelihood of) metastasis, prevent recurrence, sensitize the cancer to a chemotherapeutic, sensitize the cancer to radiotherapy, sensitize the cancer to phototherapy.

The terms “cancer stem cell” and “CSC” refer to the subpopulation of cancer cells within tumors that have capabilities of self-renewal, differentiation, and tumorigenicity when transplanted into an animal host. Compared to “bulk” cancer cells, CSCs have increased mitochondrial mass, enhanced mitochondrial biogenesis, and higher activation of mitochondrial protein translation. As used herein, a “circulating tumor cell” is a cancer cell that has shed into the vasculature or lymphatics from a primary tumor and is carried around the body in the blood circulation. The CellSearch Circulating Tumor Cell Test may be used to detect circulating tumor cells.

The phrases “ATP-high” and “ATP-low” refer to cell sub-populations having ATP-based fluorescent signals representing the upper and lower portions of the ATP-based fluorescent signals, respectively, from a starting cell population. The upper portion may represent the top 25% of the starting cell population's ATP-based fluorescent signals, or the top 20%, or the top 15%, or the top 10%, or the top 5%, or the top 2%, or the top 1%. The lower portion may represent the bottom 25% of the starting cell population's ATP-based fluorescent signals, or the bottom 20%, or the bottom 15%, or the bottom 10%, or the bottom 5%, or the bottom 2%, or the bottom 1%.

The phrase “pharmaceutically effective amount,” as used herein, indicates an amount necessary to administer to a host, or to a cell, tissue, or organ of a host, to achieve a therapeutic result, such as regulating, modulating, or inhibiting protein kinase activity, e.g., inhibition of the activity of a protein kinase, or treatment of cancer. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

Bioinformatics analysis demonstrates the role of mitochondrial ATP synthesis, in 3D anchorage-independent growth, stemness, and distant metastasis. In particular, mitochondrial ATP synthesis is a key determinant of 3D anchorage-independent growth and metastasis, using a bioinformatics approach. Existing proteomic profiling data was interrogated to compare 2D-monolayers with 3D-mammospheres, in two distinct ER(+) breast cancer cell lines (MCF7 and T47D). Overall, from 1,519 common proteins in both cell lines, 21 ATP-related proteins were found to be up-regulated in both data sets, in 3D-mammospheres. Table 1, below, shows these proteins, with accession number, and the fold change in expression in MCF7 and T47D cells (spheres versus 2-D adherent growth). Out of these 21 ATP-related proteins, 7 subunits of the mitochondrial ATP-synthase were detected, including ATP5F1B, ATP5F1C, ATP5IF1, ATPSMG, ATPSPB, ATPSPD and ATPSPO. Using Ingenuity Pathway Analysis (IPA) Software, we observed that the predicted upstream regulators of 3D anchorage-independent growth were highly conserved between the two cell lines and were specifically associated with existing IPA data sets, related to tumor growth and tumor cell proliferation.

TABLE 1
The 21 ATP-related proteins up-regulated in 3D-
mammospheres for both MCF7 and T47D cell lines.

Expr Fold Change MCF7
Expr Fold Change T47D

| | | 3D Spheres v. | 3D Spheres v. |
|---|---|---|---|
| Symbol | Accession | 2D Adh. | 2D Adh. |
| ABCF3 | B4DRU9 | 358.862 | 3.849 |
| ATP13A2 | Q8NBS1 | 560.751 | 42.918 |
| ATP13A4 | H7C1P5 | 20.517 | 25.583 |
| ATP1A1 | B7Z3V1 | 51.419 | 14.905 |
| ATP1A3 | P13637 | 229.056 | 16.789 |
| ATP1A4 | Q13733 | 440.915 | 60.871 |
| ATP1B1 | A3KLL5 | 1.913 | 10.585 |
| ATP1B3 | P54709 | 12.082 | 3.433 |
| ATP2A2 | P16615 | 20.836 | 7.006 |
| ATP2A3 | Q93084 | 78.477 | 23.288 |
| ATP2B1 | E7ERY9 | 7.655 | 2.928 |
| ATP5F1B | Q0QEN7 | 10.129 | 2.087 |
| ATP5F1C | Q8TAS0 | 1.947 | 1.634 |
| ATP5IF1 | Q9UII2 | 10.127 | 16.359 |
| ATP5MG | O75964 | 1.619 | 1.429 |
| ATP5PB | Q53GB3 | 2.515 | 4.379 |
| ATP5PD | O75947 | 2.27 | 1.436 |
| ATP5PO | P48047 | 1.92 | 1.426 |
| COX5B | P10606 | 7.692 | 1.513 |
| NDUFAB1 | H3BNK3 | 87.149 | 11.95 |
| UQCR10 | Q9UDW1 | 2.457 | 1.623 |
| UQCRH | Q567R0 | 1.662 | 2.171 |

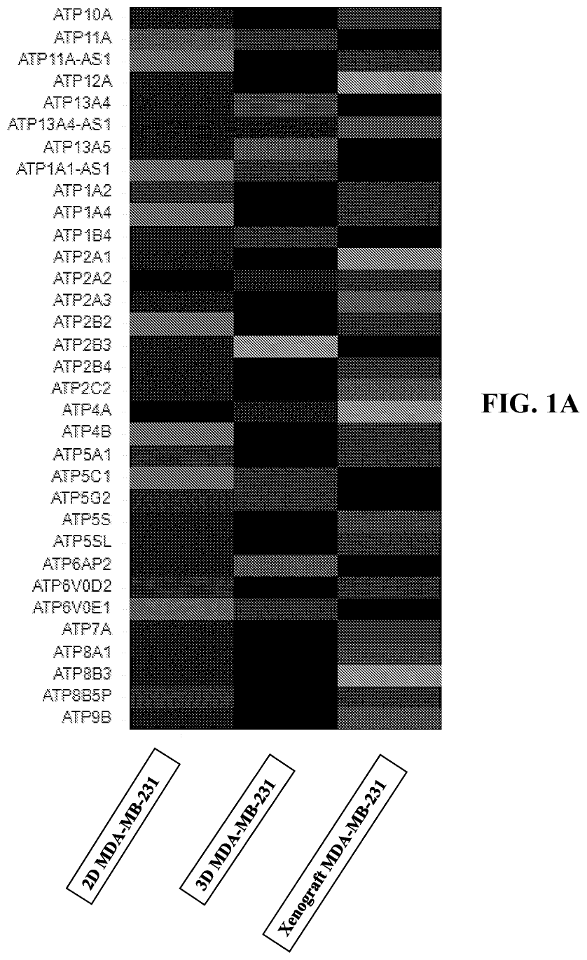

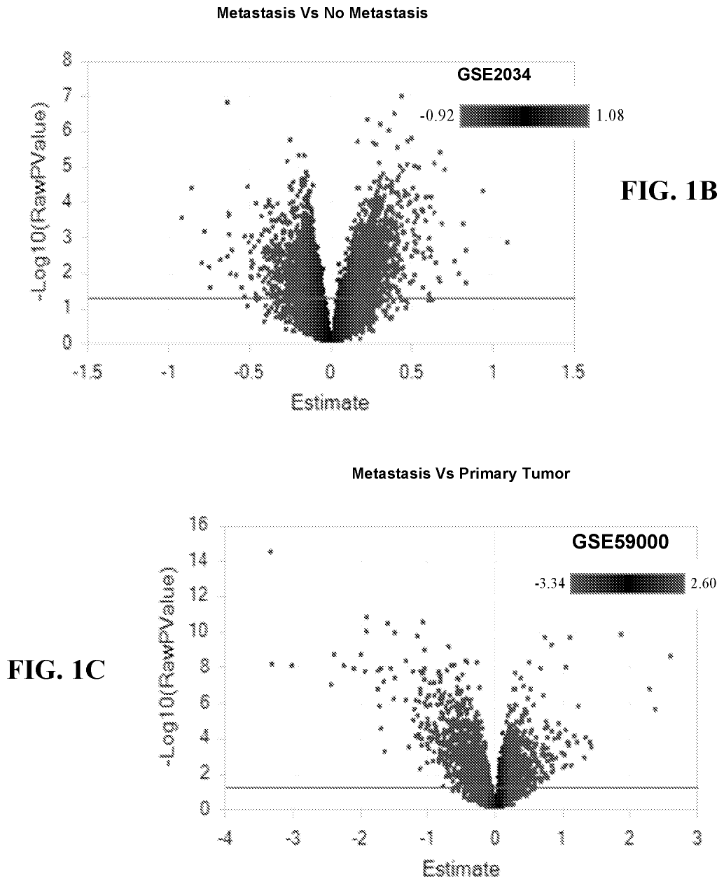

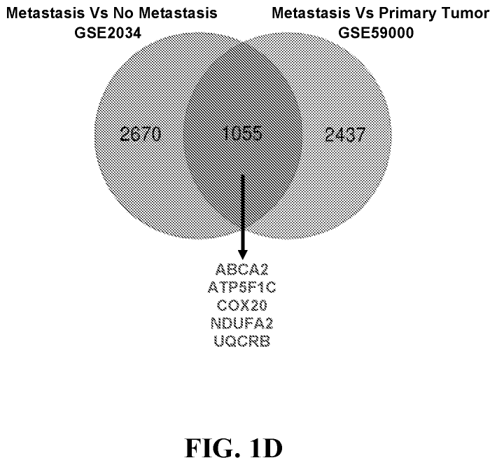

We also re-analyzed GEO transcriptional profiling data sets, comparing 2D-growth, 3D-growth, and the in vivo tumor growth of MDA-MB-231 cells (a triple-negative breast cancer cell line). FIG. 1A shows a HeatMap of ATP-related genes that were transcriptionally upregulated under both 3D growth conditions (anchorage-independent and in vivo tumors), all relative to 2D-adherent growth. The first column identifies the gene, the second column shows the expression profile in 2D MDA-MB-231 cells, the third column shows the expression profile in 3D MDA-MB-231 cells, and the fourth column shows the expression profile in xenograft MDA-MB-231 cells. Darker cells indicate less fold change, and lighter cells indicate higher fold change. The HeatMap shows the log of the fold change, e.g., the lightest cells are +/−4. In the 2D MDA-MB-231 column, lighter cells indicate a negative change (e.g., ATP11A-AS1 showed a -4 log fold change), whereas lighter cells in the 3D and xenograft columns indicate a positive change (e.g., ATP12A showed a 4 log fold change).

The transcriptional expression of ATP-related genes (OXPHOS and ATP-related transporters) in two distinct GEO DataSets related to human breast cancer metastasis were useful for identifying ATP-related genes associated with metastasis. FIGS. 1B and 1C show volcano plots for the GSE2034 and GSE59000 GEO DataSets. Specifically, FIG. 1B compares gene expression in scenarios with metastasis versus scenarios with no metastasis (GSE2034), and FIG. 1C compares gene expression in scenarios with metastasis versus the primary tumor (GSE59000). The volcano plots were produced by examining the annotations present in OncoLand Metastatic Cancer (QIAGEN OmicSoft Suite) and by performing functional “core analyses” using Ingenuity Pathway Analysis Software (IPA; QIAGEN), on genes annotated with an uncorrected p-value cut off <0.05. The transcriptional profiles of ATP-related genes (OXPHOS and ATP-related transporters), were increased and specifically associated with metastasis, in both GEO DataSets.

FIG. 1D shows a Venn diagram intersecting the two breast cancer metastasis GEO DataSets (GSE2034 and GSE59000), used to identify ATP-related genes highly upregulated in both data sets, as prognostic biomarkers of metastasis. The intersection of the two GEO DataSets was performed, as described in connection with FIGS. 1B and 1C, using IPA Software. The overlapping set of 1,055 genes contained only 5 ATP-related genes. These ATP-related genes, ABCA2, ATP5F1C, COX20, NDUFA2, and UQCRB, were highly upregulated in both metastasis GEO DataSets, and thus have prognostic value with respect to predicting metastasis of a cancer. These five ATP-related genes may be used as an ATP-related metastasis gene-signature, prognostic of metastasis. Most notably, ATP5F1C (also known as ATP5C1) encodes the gamma-subunit of the soluble Fl-catalytic core of the mitochondrial ATP synthase. UQCRB is the essential component of mitochondrial complex III, which functionally binds ubiquinone and participates in electron transport. COX20 is a chaperone that is essential for the assembly of mitochondrial complex IV. NDUFA2 is essential for the function of mitochondrial complex I. Finally, ABCA2 is a member of the ATP-binding cassette transporter gene family.

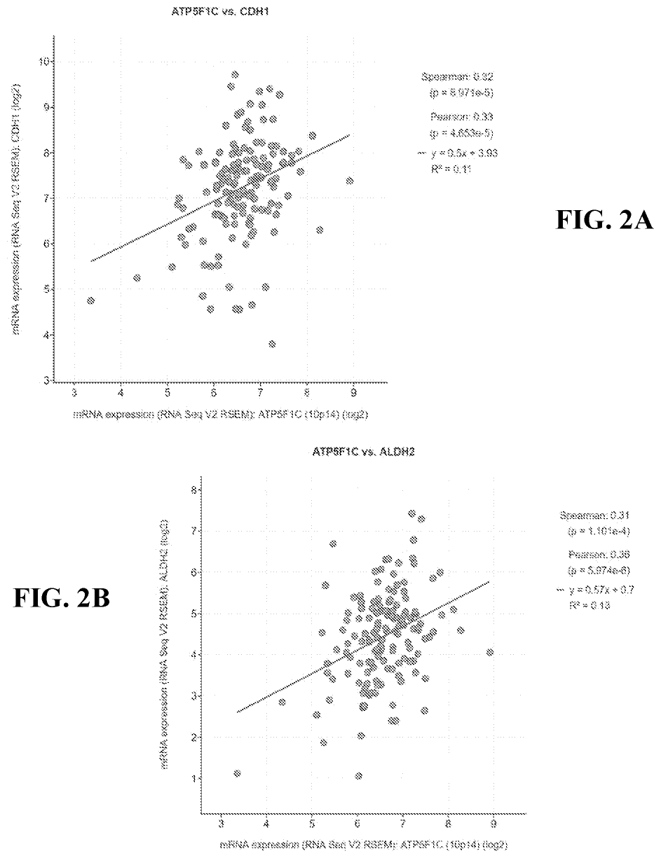

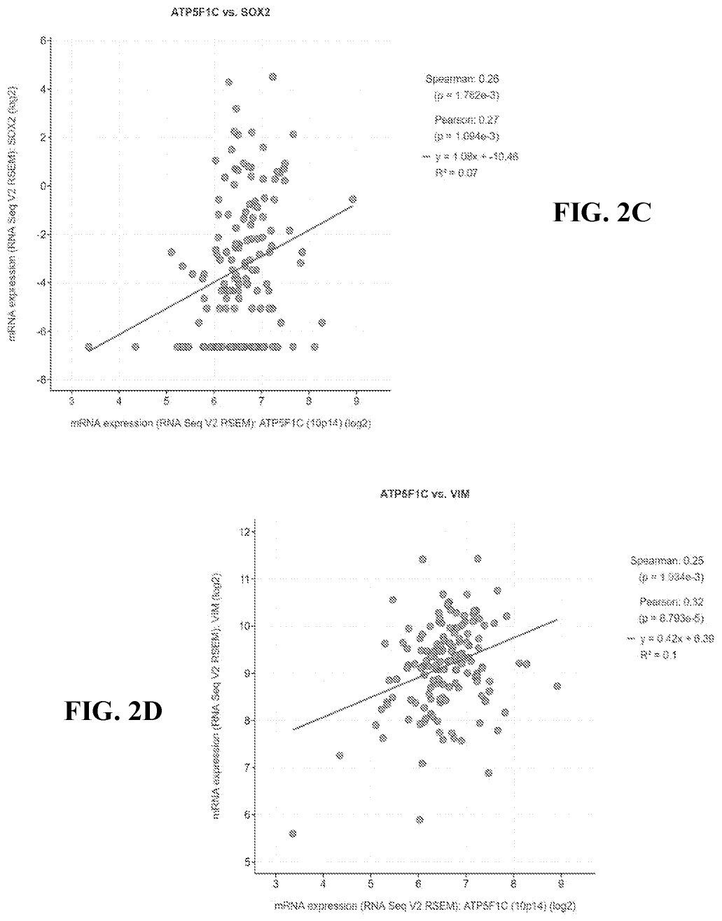

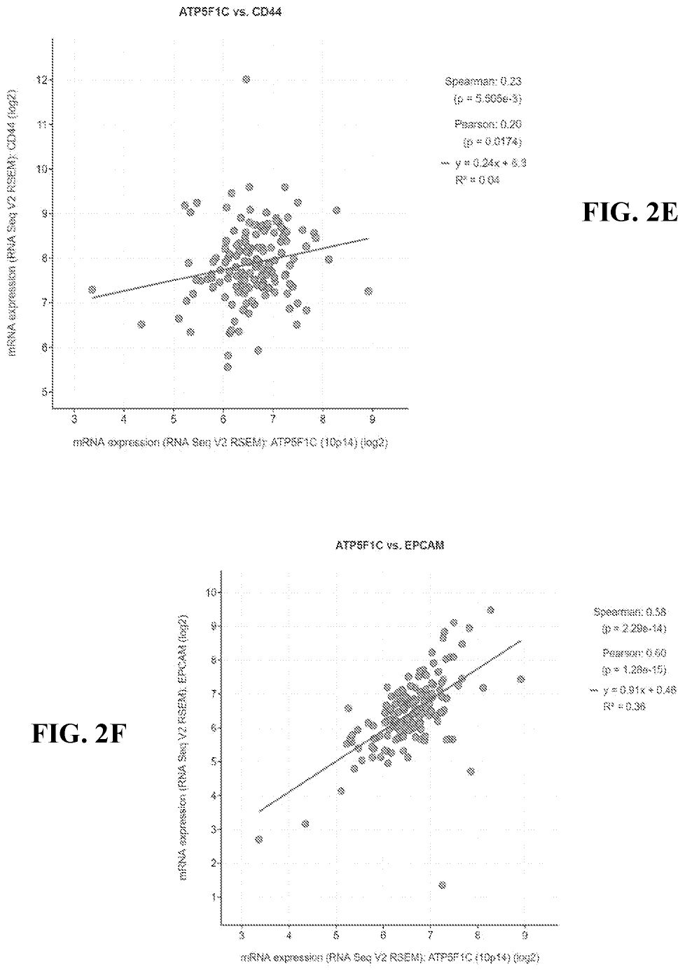

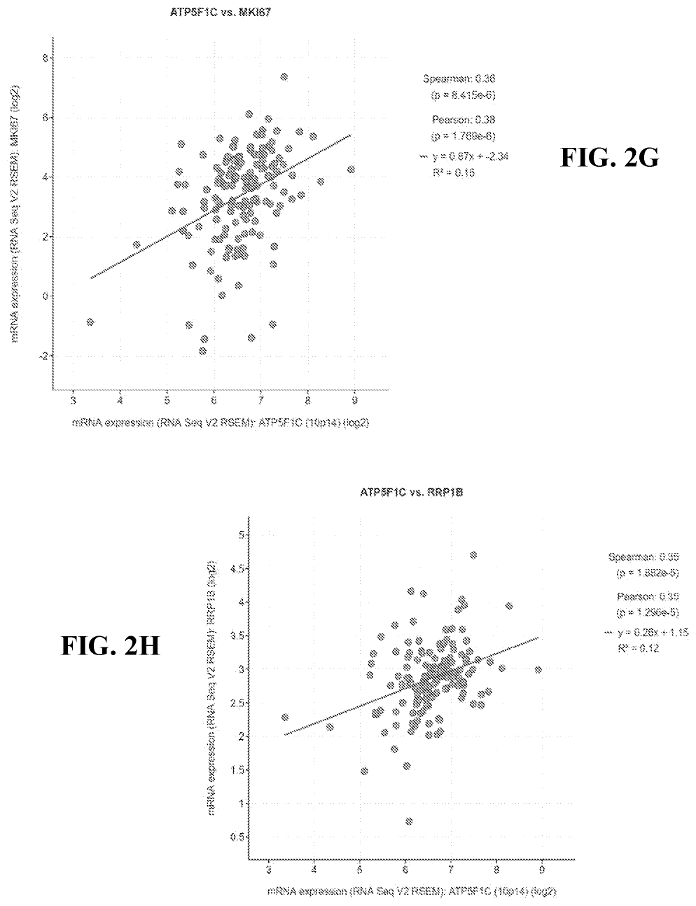

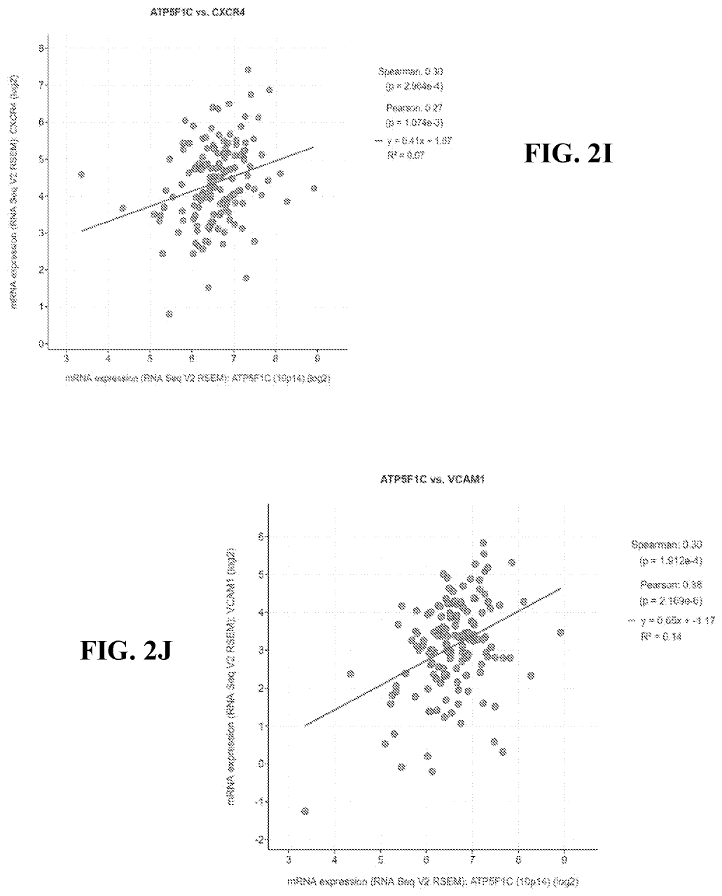

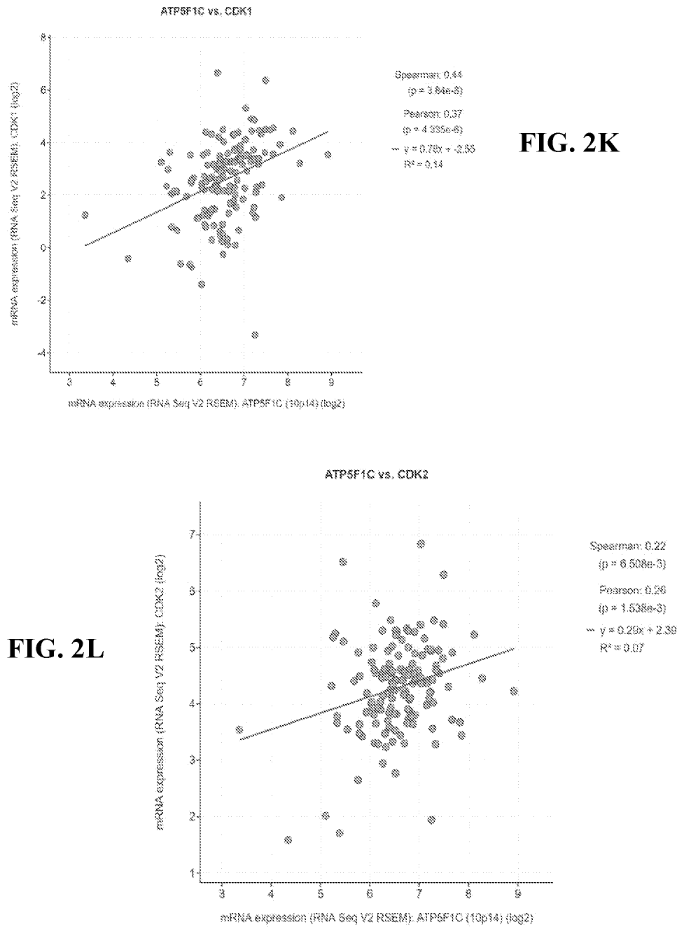

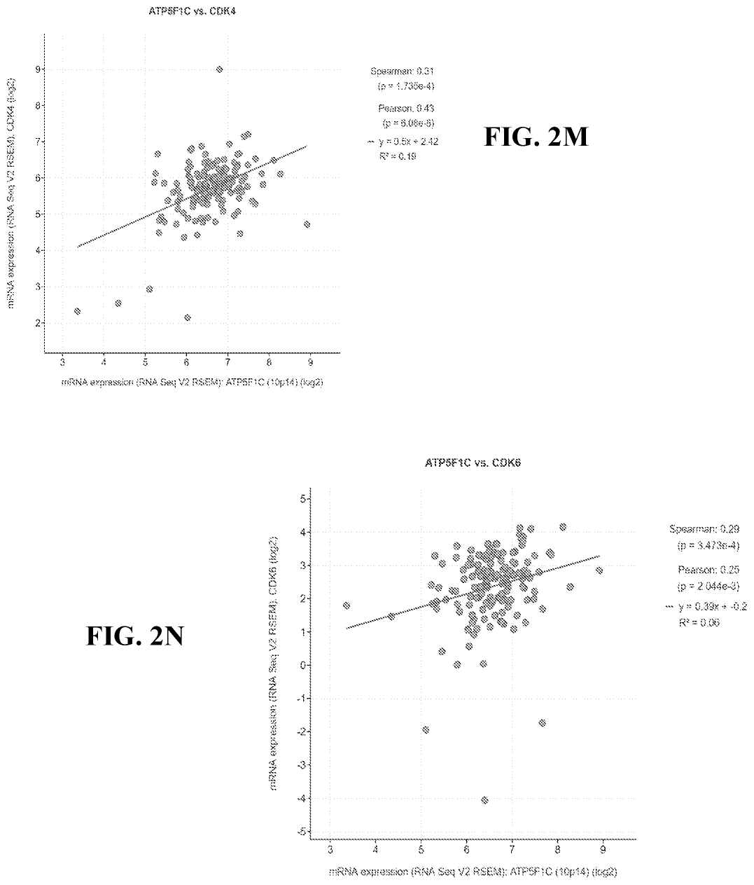

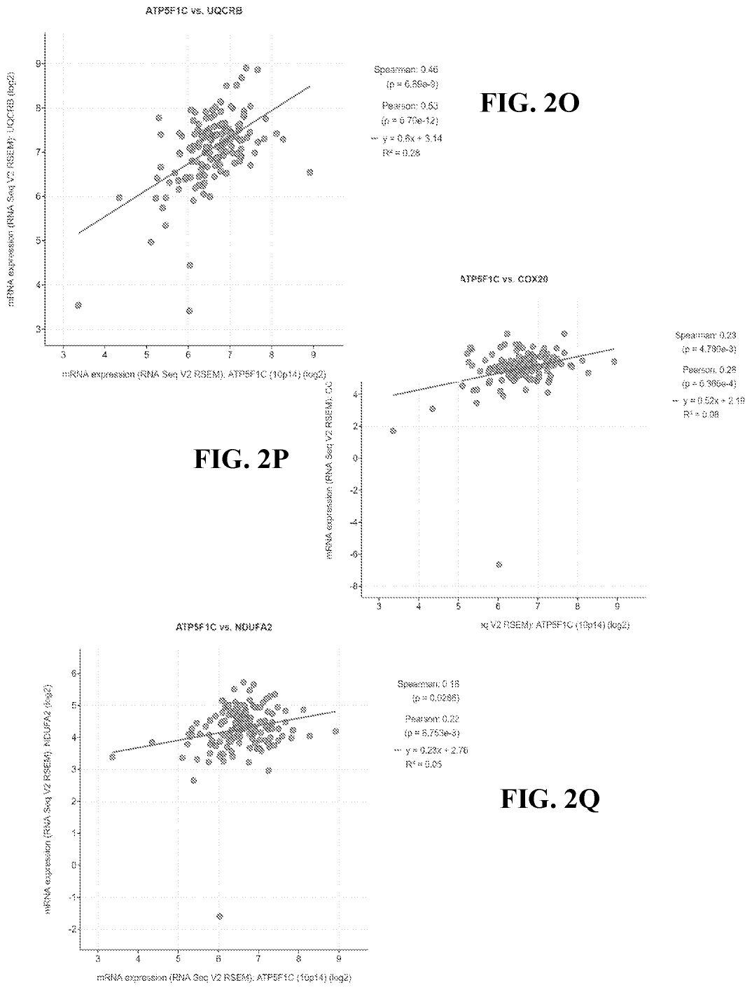

In bona fide breast cancer metastatic lesions, ATP5F1C transcriptional expression is positively correlated with the co-expression of: i) five metastatic marker genes (EPCAM, MKI67, RRP1B, VCAM1, CXCR4); ii) four cell cycle regulatory genes (CDK1, CDK2, CDK4, CDK6); and iii) eleven CSC marker genes (CDH1, ALDH2, ALDH1BA1, ALDH9A1, SOX2, VIM, CDH2, ALDH7A1, ALDH1B1, CD44, ALDH3B2, listed in rank order of statistical significance). FIGS. 2A-2N are data plots showing the positive correlation of APT5F1C versus each of these genes, in the order of CDH1, ALDH2, SOX2, VIM, CD44, EPCAM, MKI67, RRP1B, CXCR4, VCAM1, CDK1, CDK2, CDK4, and CDK6.

Additionally, ATP5F1C transcriptional expression is also positively correlated with the co-expression of mitochondrial complexes I-V, mt-DNA encoded transcripts and three other members of the five-member metastasis gene-signature, namely UQCRB, COX20 and NDUFA2. FIGS. 20-2Q are data plots showing the positive correlation of APT5F1C versus UQCRB, COX20, and NDUFA2, respectively. The expression of two members of this metastasis gene signature, ATP5F1C and UQCRB, has been functionally correlated with maximal oxygen uptake (Vo2max) and a high percentage of type 1 fibers (mitochondrial-rich) in human skeletal muscle tissues.

The expression of ATP5F1C in skeletal muscle is also increased significantly after exercise training, reflecting increased muscle fitness in patients. Conversely, ATP5F1C levels decreased with advanced age and were reduced in progeria syndrome patients. These results are highly suggestive that high ATP5F1C expression is a biomarker of increased mitochondrial ATP production at the cellular level.

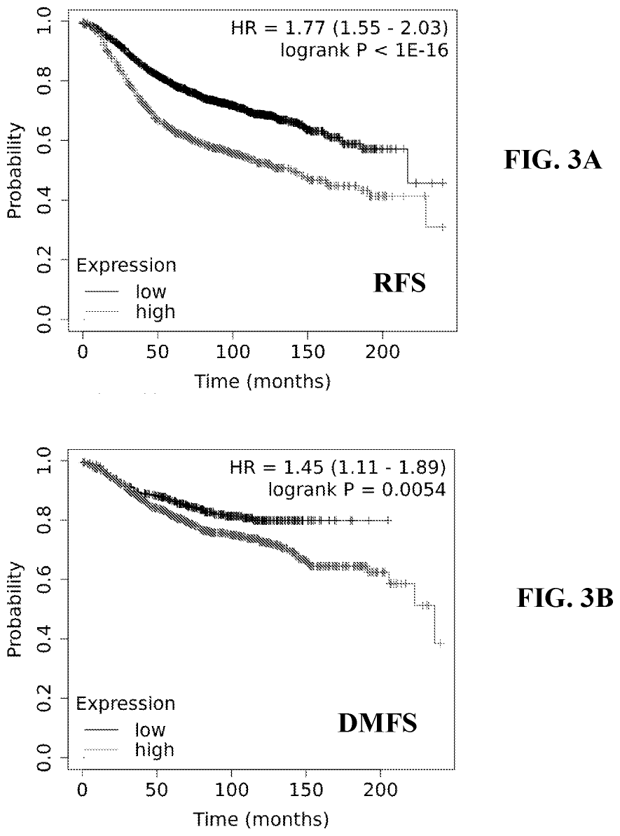

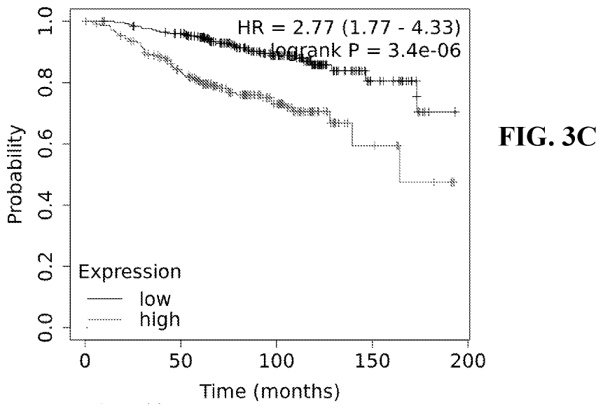

Using Kaplan-Meier (K-M) analysis, we determined that ATP5F1C is a prognostic biomarker for distant metastasis and tumor recurrence, especially in ER(+) patients that are lymph node negative at diagnosis and were treated with Tamoxifen (Hazard Ratio (recurrence-free survival)=2.77; P=3.4E-06; N=471). FIG. 3A shows the Kaplan-Meier curve for ER(+), recurrence-free survival (N=3,082), FIG. 3B shows the Kaplan-Meier curve for ER(+), distant metastases-free survival (N=1,395), and FIG. 3C shows the Kaplan-Meier curve for ER(+), lymph node negative, Tamoxifen-treated, relapse-free survival (N=471).

These results are consistent with previous studies showing that mitochondrial activity is functionally upregulated in breast cancer metastatic lesions, within surgically excised lymph nodes, using a histochemical activity stain that detect mitochondrial complex IV. In addition, the inventors previously noted that 16 members of the ATPS gene family, including ATP5F1C (4.64-fold; p=1.14E-05), are transcriptionally upregulated in human breast cancer cells, relative to adjacent stromal cells, in samples derived from N=28 breast cancer patients.

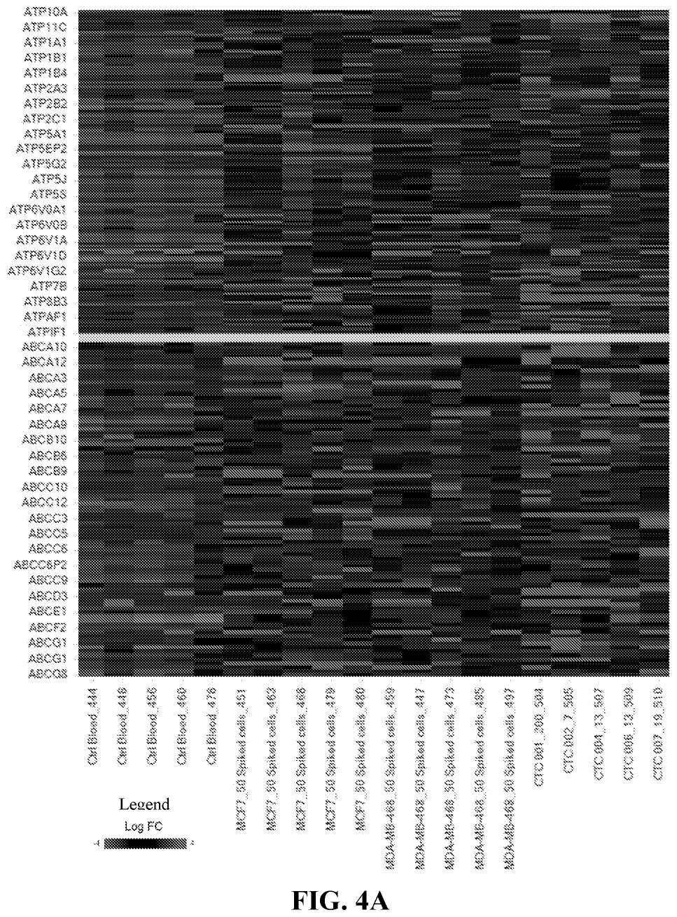

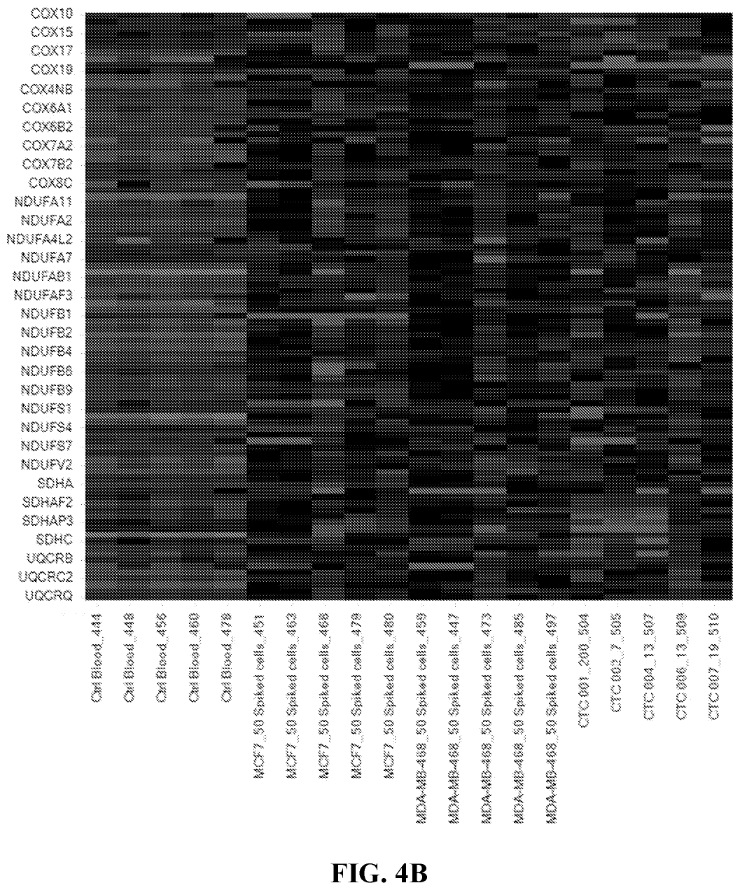

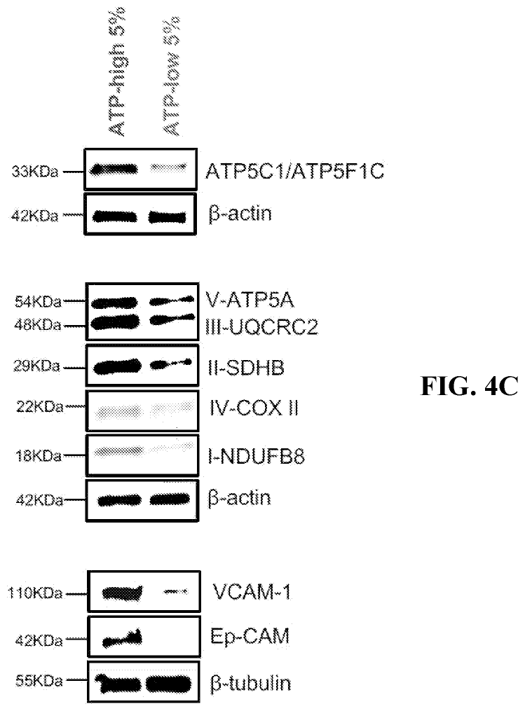

Existing GEO DataSets (GSE55470) were used to assess the use of ATP-related genes and OXPHOS genes as transcriptional biomarkers of breast cancer circulating tumor cells (CTCs) in patients. FIG. 4A shows a HeatMap of the ATP-ABC gene expression profile in the data set, and include a legend. FIG. 4B shows a HeatMap of the OXPHOS gene expression profile, based on the same legend in FIG. 4A. Generally, the lighter the cell, the higher the absolute value of the expression. Distinguishing between positive and negative fold changes is difficult in black and white used in connection with this application. The majority of cells the first five columns, for the control blood, are green in the original HeatMap, indicating a negative fold change in expression. Cells in the majority of the remaining columns are red, indicating a positive fold change in expression. Overall, the data demonstrate that high ATP content in CTCs may be useful as a biomarker, to identify and track CTCs in whole blood, thereby potentially improving cancer diagnosis and preventing metastatic spread.

FIG. 4C shows the results of a Western blot analysis of MDA-MB-231 cells in the ATP-high and ATP-low sub-populations. The results show that mitochondrial markers and CTC markers are both upregulated in ATP-high MDA-MB-231 cells. Mitochondrial markers from Complexes I to V, including ATP5F1C, were all over-expressed in MDA-MB-231 cells in the ATP-high sub-population, relative to MDA-MB-231 cells in the ATP-low sub-population. In addition, two known markers of CTCs and metastasis (VCAM-1 and Ep-CAM) were over-expressed in MDA-MB-231 cells in the ATP-high sub-population. Beta-actin and Beta-tubulin were used as markers for equal protein loading.

Taken together, the bioinformatics data and analysis shows that increased mitochondrial ATP synthesis could be a key driver of 3D anchorage-independent growth and metastasis. Based on this analysis, cancer cells having the highest levels of ATP would be highly proliferative, more stem-like, undergo 3D-anchorage independent growth and would possess other aggressive behaviors, as compared to cancer cells with lower levels of ATP. Likewise, those cells having the lowest levels of ATP would be more dormant. Both sub-populations have considerable value for, among other uses, cancer research and drug screening. The present approach provides methods for separating these sub-populations from cancer cell populations through metabolic fractionation. Cancer cell populations have numerous sub-populations. CSCs are a small sub-population of cancer cells having self-renewal properties, are capable of differentiation, and they show tumorigenicity when transplanted. As described herein, however, not all CSCs are created equal. CSCs separated and purified on the basis of ATP levels have unique phenotypic properties not found in naturally-occurring cancer cell populations, or even in CSCs separated and purified using convention markers such as CD44, CD24, and CD133.

Under the present approach, cells, and preferably cancer cells, may be fractionated based on metabolic condition using a fluorescent ATP-labeling dye, such as ATP-Red 1, and flow cytometry. ATP levels ultimately determine the phenotypic traits of cancer cells, such as “stemness” and proliferation capacity. The ATP-labeling dye can thus be used to identify and purify the energetically “fittest” cancer cells from within the total cell population. The inventors selected Biotracker ATP-Red 1, a fluorescent vital dye, to label ATP in living cancer cells. It should be appreciated that other fluorescent ATP imaging probes, including later-developed probes, may be used without departing from the present approach. Preferably, the fluorescent ATP imaging probe targets mitochondrial ATP.

ATP-Red 1 is normally non-fluorescent, but becomes fluorescent when bound to ATP, but not to any other related nucleotides or metabolites, including ADP. More specifically, BioTracker ATP-Red 1 does not recognize sugars (arabinose, galactose, glucose, fructose, ribose, sorbose, sucrose or xylose) or other nucleotides (AMP, ADP, CMP, CDP, CTP, UMP, UDP, UTP, GMP, GDP or GTP). Importantly, this fluorescent ATP imaging probe exhibits a “turn-on” fluorescence-response toward ATP, with a near 6-fold fluorescence enhancement. Using fluorescence microscopy, ATP-Red 1 is predominantly detected within mitochondria, the major source of cellular ATP production. Therefore, ATP-Red 1 is preferred as a fluorescent probe to metabolically fractionate the cancer cell population by flow cytometry.

The cancer cell population may be separated or fractionated into ATP-high and ATP-low cell sub-populations, and then subjected to phenotypic characterization. The sub-populations may be defined term terms of a percentage of the top and bottom fluorescent signals (e.g., top and bottom 20%, top and bottom 1%, etc.), and the FACS gate cut-offs for cell selection and collection are determined based on the selected percentages. The data disclosed herein primarily relied on the top and bottom 5%, and the top and bottom 10% as the gate cut-offs, but it should be appreciated that other percentages may be used without departing from the present approach. Of course, the percentage should be less than 50%, and it should be expected that the larger the percentage, the less specific the phenotypic characterization will be for a given cell population.

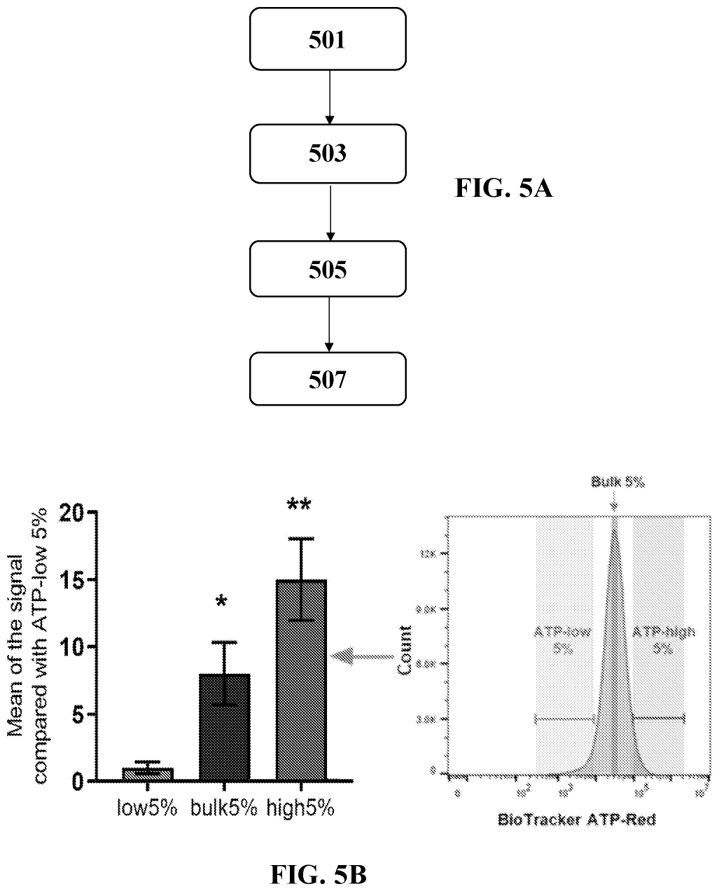

FIG. 5A illustrates an embodiment of the metabolic fractionation procedure according to the present embodiment. The fluorescent ATP imaging probe may be dissolved in media and incubated 501. The results described herein involved 5 μM Biotracker ATP-Red as the fluorescent ATP imaging probe, dissolved in media and incubated in cells for 30 minutes. The cells were then washed with PBS and trypsinized, and re-suspended in a FACS buffer and passed through a 40 μm cell strainer 503. Cells derived from 3D spheres or 2D adherent condition were analyzed using a FACS sorter instrument (e.g., SONY SH800) 505. Cells were gated at the desired ATP content, using ATP-based fluorescent signals (e.g., top/bottom 1%, 2%, 3%, 4%, 5%, 10%, etc.) and sorted 507. FIG. 5B illustrates an example of metabolic fractionation of MCF7 cells with ATP-Red 1, to isolate ATP-high (top 5%) and ATP-low (bottom 5%) cell sub-populations. The bulk (5%) population was also selected for comparison purposes. The right image shows the cell count at various fluorescent intensities, and identifies the regions of the ATP-high (top 5%) sub-population, the ATP-low (bottom 5%) sub-population, and the bulk median. The left image of FIG. 5B shows the mean ATP-based fluorescent signal for each sub-population. Based on mean signal intensity, we estimate that ATP-high MCF7 cells have approximately 15-fold higher levels of ATP, as compared with the ATP-low population; and 2-fold higher levels of ATP, as compared with the bulk cell population.

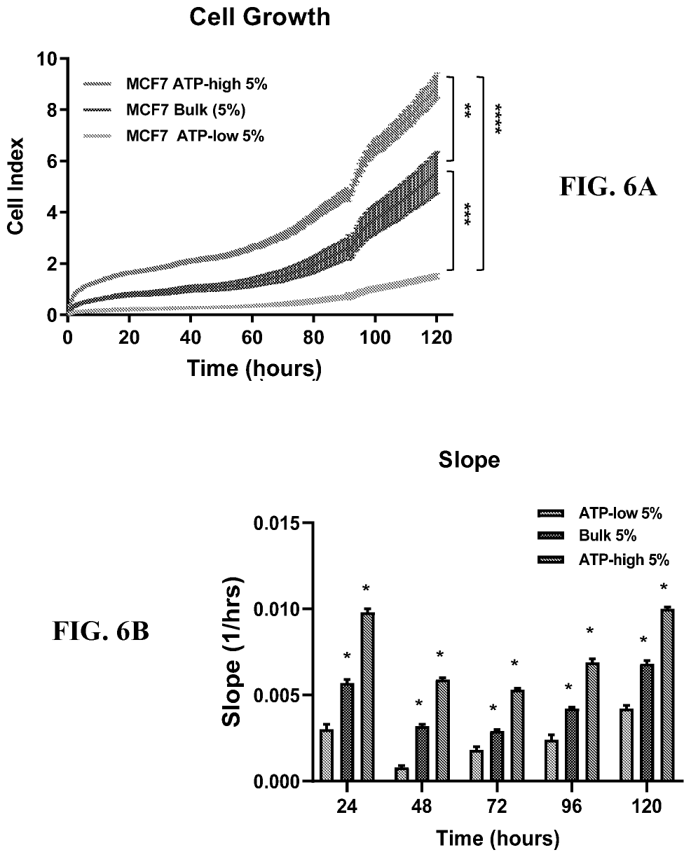

FIGS. 6A and 6B show results from a continuous, real-time assay system on cell proliferation of all three cell sub-populations (ATP-low 5%, Bulk 5%, ATP-high 5%). Cell proliferation was assessed using the xCELLigence® RTCA DP instrument. Cells were first sorted for ATP content, counted and seeded (1×10⁴ in common media) in RTCA DP E-Plates for real-time growth analysis. Graphically, the 3 sub-populations (ATP-low 5%, Bulk 5%, ATP-high 5%) are all represented. The results indicate that the ATP-high population is approximately 2-fold more proliferative, relative to the bulk cell population and approximately 5-fold more proliferative, relative to the ATP-low population, after 120 hours. Data represent the mean±SD, n=3. One-way ANOVA, Dunnett's multiple comparisons test, **p<0.001, ***p<0.001, ****p<0.0001. As can be seen in FIG. 6A, the ATP-high sub-population had a significantly higher cell index compared to the other sub-populations, and the ATP-low sub-population and a significantly lower cell index compared to the other sub-populations. FIG. 6B shows the slope of the cell index over time for each sub-population. At each time point (24, 48, 72, 96 and 120 hours) the slope of the ATP-high cell population, was significantly higher compared to the other 2 sub-populations. Data represent mean±SD, n=3. Two-way ANOVA, Tukey corrected, *p<0.01. It is apparent that the ATP-high sub-population had the highest rate of proliferation across the entire 120-hour assessment. The results indicate that the ATP-high population at least, approximately, 2-fold more proliferative, relative to the bulk cell population, and at least, approximately, 5-fold more proliferative, relative to the ATP-low population. This demonstrates mitochondrial ATP levels are a key determinant of MCF7 cell proliferation, and that the metabolic fractionation with a fluorescent ATP imagine probe of the present approach is an effective technique for identifying the most proliferative, and least proliferative, cell sub-populations.

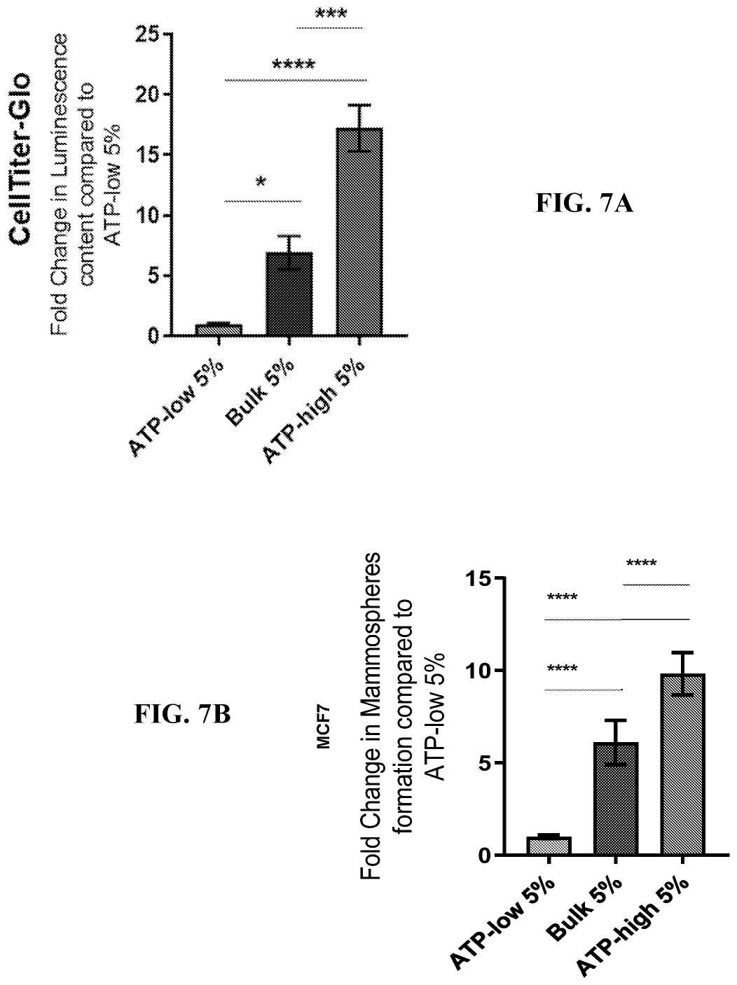

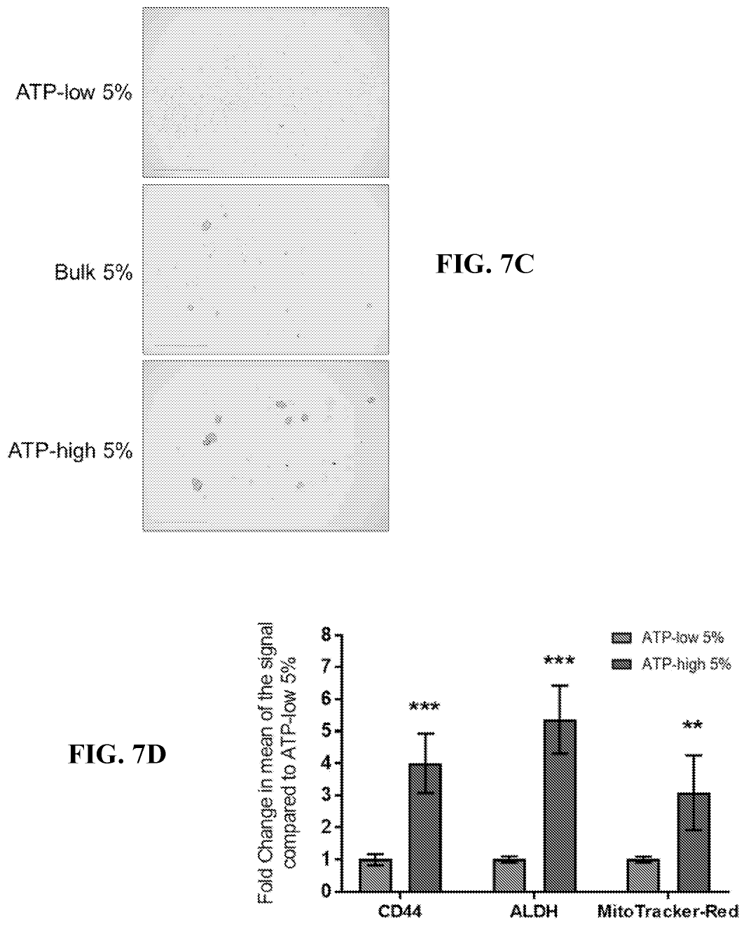

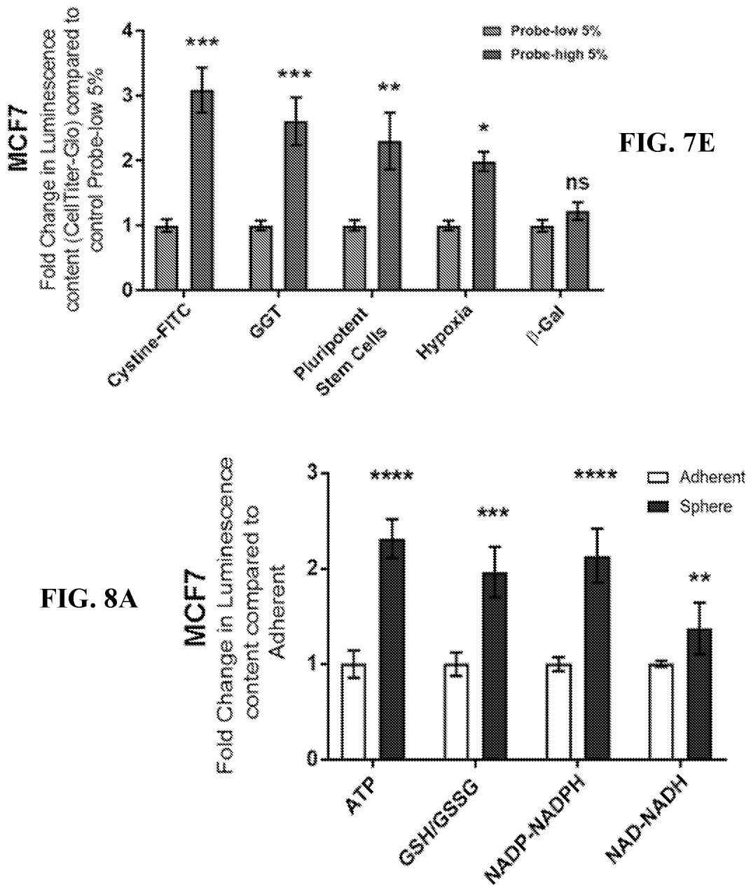

Further assays confirmed that the ATP-high MCF7 sub-population are energetically hyper-proliferative, have significantly increased 3D anchorage-independent growth, cancer stem cell markers, and mitochondrial mass. To confirm the selectivity of ATP-Red 1, Cell-Titer-Glo was used to measure ATP levels in cells, after flow cytometry. However, as Cell-Titer-Glo is a luciferase-based assay, it requires cell lysis to detect ATP levels and as a consequence, it cannot be used for live cell sorting or imaging. After cell counting, equal numbers of single cells were then used to evaluate their relative ATP content by luminescence, using the Varioskan™ LUX plate reader. FIG. 7A is a bar graph that shows cells in the ATP-high MCF7 sub-population have at least a 15-fold increase in ATP levels, while bulk cells showed about a 7-fold increase in ATP, relative to the ATP-low cell population. This also shows that the ATP-high sub-population has at least twice the ATP level of the bulk cells in the MCF7 population.

The 3D-mammosphere assay was used to measure anchorage-independent growth, which is a functional read-out for CSC activity and CSC propagation. FIG. 7B shows results of the 3D-mammosphere assay for the ATP-high, bulk, and ATP-low sub-populations, using 5% as the gate cutoff. FIG. 7C shows comparative images of the cell sub-populations after the assay. Images of 3D mammospheres were acquired using the EVOS FL Auto2 microscope. The panels represented the 3 sorted cell population cells. Representative Images are shown. A 4X objective was used. Scale bar =1,000 μm. The ATP-high MCF7 cell sub-population showed a 9-fold increase in 3D spheroid formation relative to the ATP-low sub-population, and nearly double the mammosphere formation of the bulk sub-population. These data indicate that ATP-high cells would be better able to undergo 3D anchorage-independent growth than the bulk CSC population.

Two well-established CSC markers, CD44 and ALDH activity, were used to examine the “stemness” of the sub-populations. FIG. 7D shows that the ATP-high MCF7 cell sub-population (right bars) was enriched nearly 4-fold in CD44 cell surface expression and about 5.5-fold in ALDH-activity, when using a FACS gating cut-off of 5%, compared to the ATP-low sub-population (left bars). Similar results were also obtained with MitoTracker-Deep-Red, a well-established marker of mitochondrial mass, which revealed a 3-fold increase in the ATP-high MCF7 sub-population compared to the ATP-low sub-population. Mitochondrial mass is a specific marker of stemness in CSCs.

These demonstrate that the metabolic fractionation of the present approach enriches the CSC activity in the ATP-high sub-population. Importantly, high ALDH activity is considered to be a biomarker of the EMT (epithelial-mesenchymal transition) in CSCs, whereas CD44 is considered to be more of an epithelial CSC marker. So, both epithelial and mesenchymal CSCs are significantly enriched in the ATP-high cell sub-population.

Fluorescent vital probes for anti-oxidant capacity and pluripotency also select for a population of ATP-high MCF7 cells. The effectiveness of the BioTracker ATP-Red 1 imaging probe was compared with several other fluorescent vital dyes, specifically for ATP-high cell population selectivity. For this purpose, MCF7 cell 2D monolayers were harvested with trypsin and lived-stained with a panel of 5 other fluorescent BioTracker probes for i) anti-oxidant capacity, including cystine uptake (“cysteine-FITC”) and gamma-glutamyl-transpeptidase activity, or GGT; ii) pluripotent stem cells; iii) hypoxia; and iv) senescence (beta-galactosidase activity, or β-Gal). Then, total ATP levels were determined using Cell-Titer-Glo, immediately following flow cytometry.

FIG. 7E shows the results of the Cell-Titer-Glo of this analysis, showing the fold change in luminescence of the highest 5% (the right bar for each probe) relative to the lowest 5% (the left bar for each probe). Remarkably, the probes for anti-oxidant capacity (cystine uptake and GGT activity), as well as pluripotency, all selected for the ATP-high sub-population of MCF7 cells. However, of the additional fluorescent vital probes tested, the BioTracker probe that directly measures the uptake of cystine-FITC, was the most effective at selecting the ATP-high cell sub-population, but it was not as effective as ATP-Red 1 (3-fold vs. 20-fold). Interestingly, high anti-oxidant capacity is known to be strictly associated with stemness and the drug-resistance phenotype. The hypoxia probe also positively selected the ATP-high cell sub-population. This may be due to the association between hypoxia and increased mitochondrial biogenesis. However, the senescence probe (beta-galactosidase activity) did not select for either the ATP-high or the ATP-low cell population.

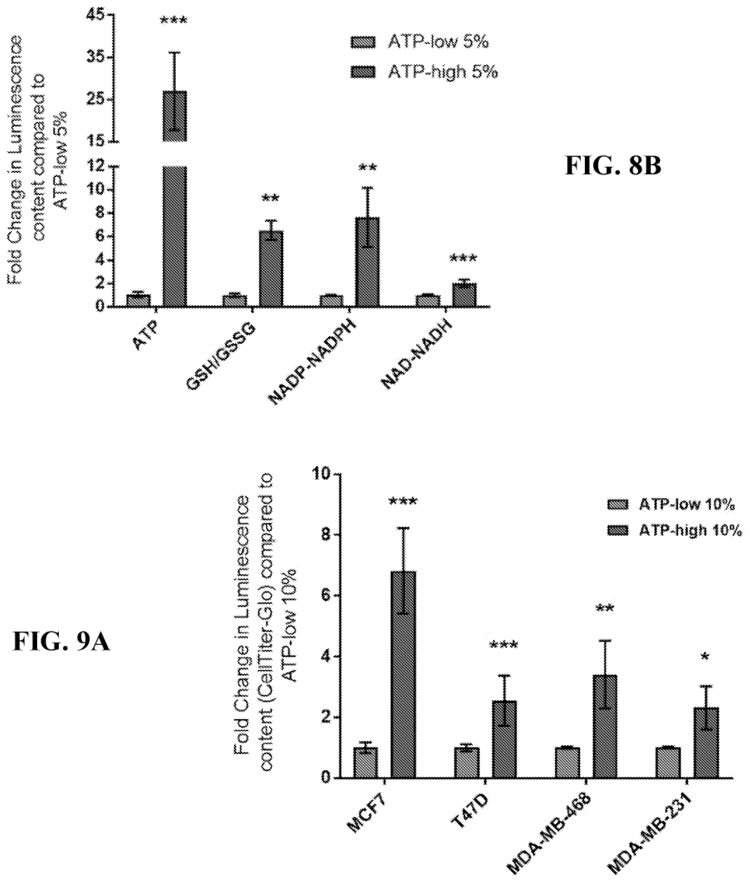

FIGS. 8A and 8B show results relating to the metabolic profiling of 3D-mammospheres and ATP-high MCF7 cells. The intracellular ATP levels in MCF7 cells, cultured either as 2D monolayers or 3D spheroids, were compared to better understand the metabolism underlying 3D-anchorage independent growth. The latter cell population is known to be highly-enriched in CSCs. Metabolite levels in MCF7 cells grown as 2D adherent monolayers or 3D mammospheres were compared, using Promega kits (Cell-Titer-Glo, GSH/GSSG-Glo, NADP-NADPH-Glo, NAD-NADH-Glo). 2D monolayers and 3D mammospheres were first dissociated into single cells with trypsin, syringed with a 25-gauge needle and passed through a 40-μm cell strainer. After cell counting, equal numbers of single cells were then used to evaluate their relative luminescence content. Note that cells derived from 3D mammospheres showed over a 2-fold increase in ATP levels; a near 2-fold increase in reduced glutathione levels; over a 2-fold increase in NADP-NADPH levels and near 1.5-fold increase in NAD-NADH levels, all relative to 2D monolayers. Data represent the mean fold increase over adherent cells±SD, n=4. Unpaired t-test, ** p<0.005, ***p<0.0005, ****p<0.0001.

FIG. 8A compares the change in luminescence of 3D spheroids (right bar) to 2D monolayers (adherent, left bar) for probes targeting ATP, GSH/GSSG, NADP-NADPH, and NAD-NADH. Quantitative analysis of MCF7 cells derived from 3D spheroids showed a 2.3-fold increase in ATP levels, relative to 2D monolayer cells. Approximately 2-fold increases in both the GSH/GSSG ratio and NADP/H levels were observed, and similar results were obtained with NAD/H. These data are consistent with the idea that 3D anchorage-independent growth may also require increased anti-oxidant capacity.

Given the foregoing, an ATP-high sub-population of 2D monolayer cells are expected to have an ability to undergo 3D anchorage-independent growth. Under conditions of low-attachment, >90% of MCF7 cells normally undergo anoikis, a specialized form of apoptotic cell death. Higher ATP levels would presumably allow CSCs to better resist the high stress of growth in suspension, caused by the absence of cell-substrate attachment. However, higher energy reserves might also confer resistance to multiple stressors, resulting in multi-drug resistance.

ATP-high and ATP-low MCF7 cells were subjected to metabolic profiling for NAD/H and two key anti-oxidants, GSH and NADP/H using Promega kits (Cell-Titer-Glo, GSH/GSSG-Glo, NADP-NADPH-Glo, NAD-NADH-Glo). Cells in 2D monolayers were first stained with BioTracker ATP-Red 1 and sorted by ATP content by flow cytometry. After cell counting, equal numbers of single cells were then used to evaluate their relative luminescence content. Note that ATP-high cells showed a near 25-fold increase in ATP levels; a 6-fold increase in reduced glutathione levels; a near 8-fold increase in NADP-NADPH levels and >2-fold increase in NAD-NADH levels, all relative to ATP-low MCF7 cells. Data represent the mean fold increase over ATP-low 5% cells±SD, n=4. Unpaired t-test, ** p<0.005, ***p<0.0005.As shown in FIG. 8B, cells in the ATP-high sub-population contain over 1.5-fold more NAD/H, over 7.5-fold more NADP/H, and over 7-fold more reduced glutathione (GSH/GSSG ratio), all relative to cells in the ATP-low sub-population. These data show that MCF7 cells in the ATP-high sub-population are more energetic and, as consequence, they fortify their anti-oxidant capacity. High levels of anti-oxidants are known to be associated with drug-resistance in cancer cells, indicative of a multi-drug resistant phenotype. Cells in the ATP-high MCF7 sub-population thus mimic the 3D metabolic phenotype, demonstrating that the present approach of separating ATP-high cells from a population produces a unique phenotype, having numerous potential uses.

The ATP-high and ATP-low sub-population phenotypes exist across numerous cancer types. ATP-high sub-populations of MCF7, T47D, MDA-MB-231 and MDA-MB-468 cells all show increased 3D anchorage-independent growth. Compositions of ATP-high and ATP-low cell sub-populations from three other human breast cancer cells lines, T47D, MDA-MB-231 and MDA-MB-468 cells, prepared with a FACS gating cut-off of 10%. The relative amount of ATP in the ATP-high and ATP-low cell sub-populations, was independently validated using Cell-Titer-Glo. 2D monolayer cells were first stained with BioTracker ATP-Red 1 and sorted by ATP content, using a flow cytometer. After cell counting, equal numbers of single cells were then used to evaluate their relative luminescence content. In this series of experiments, we used a cut-off of 10% to define the ATP-high and ATP-low cell populations. Note that this metabolic fractionation scheme can be successfully applied to other breast cancer cell lines. Data represent the mean fold increase over ATP-low 10% cells±SD, n =3. Unpaired t-test, * p<0.05, ** p<0.005, ***p<0.0005.

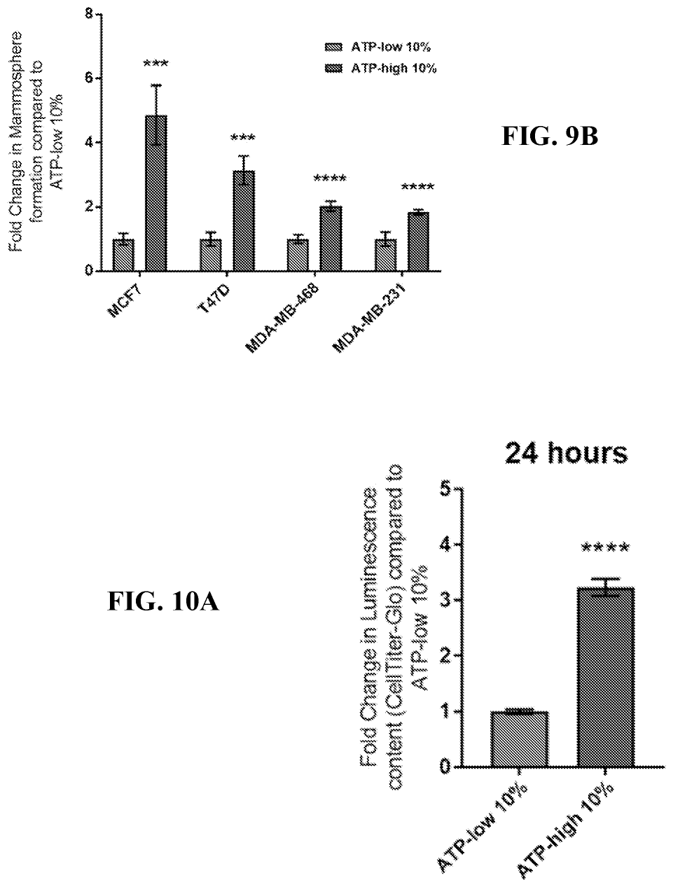

FIGS. 9A and 9B show Cell-Titer-Glo and 3D mammosphere formation results for ATP-high and ATP-low sub-populations of MCF7, T47D, MDA-MB-231 and MDA-MB-468 cells, using a 10% gate. FIG. 9A illustrates that the ATP-high sub-populations of all these cell lines showed increases in ATP characteristic of the ATP-high sub-population phenotype, as confirmed using the luciferase-based Cell-Titer-Glo assay, with a 2-to-3-fold increase in total ATP levels. As seen in FIG. 9B, similar results were obtained with the 3D spheroid assay, indicative of an increase in CSC activity and propagation between 1.75- and 3-fold, depending on the cell line examined. Cells in 2D monolayers were first stained with BioTracker ATP-Red 1 and sorted by ATP content, using a flow cytometer. After cell counting, 5×10³ cells were seeded onto poly-HEMA coated 6-well plate and counted after 5 days. Note that the ATP-high cell population of MCF7, T47D, MDA-MB-231 and MDA-MB-468 cells all showed an increased capacity for 3D anchorage-independent growth. Data represent the mean fold increase over ATP-low 10% cells±SD, n=3. Unpaired t-test, *** p<0.0005, ****p<0.0001.

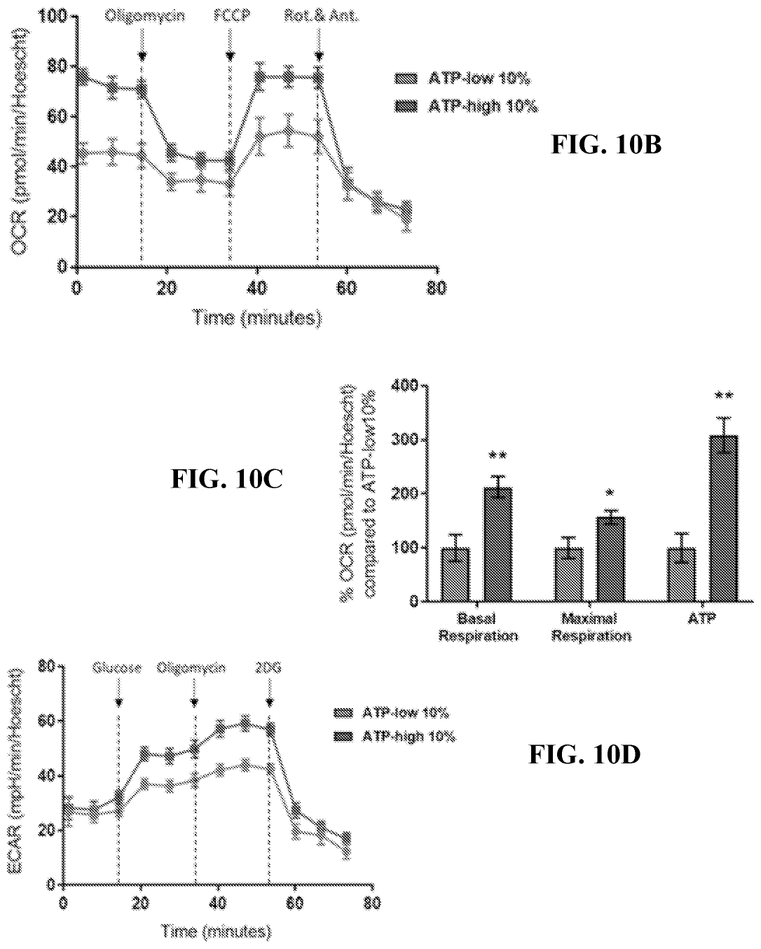

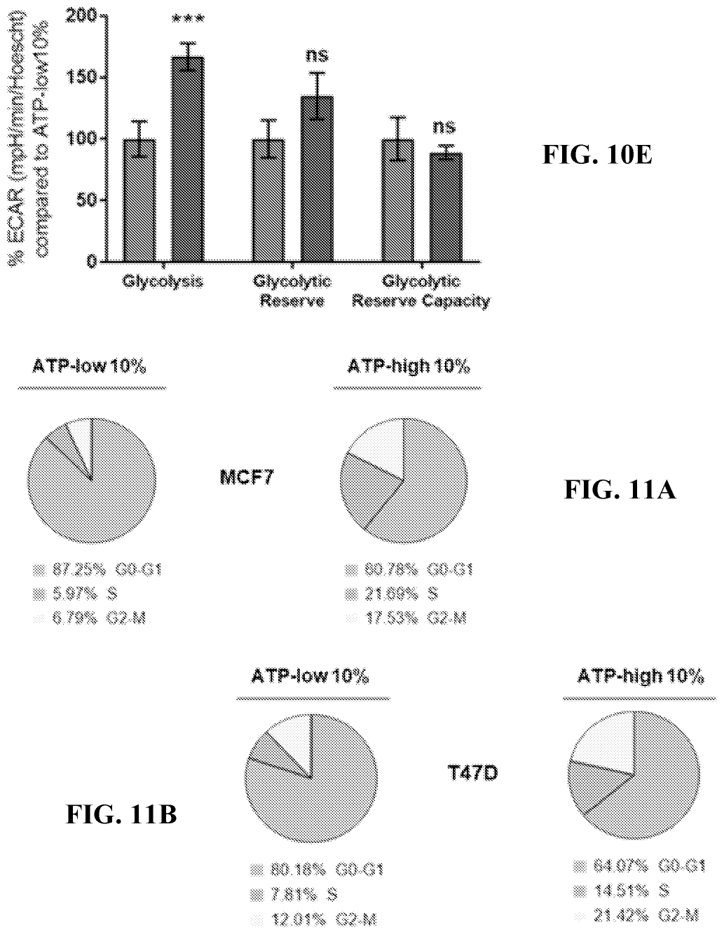

Cells in the ATP-high sub-populations show increases in oxidative mitochondrial metabolism, glycolytic rates and cell cycle progression. FIG. 10A shows luminescence in ATP-high and ATP-low sub-populations (10%) in a MCF7 cell population after a 24-hour period. After cell counting, equal numbers of single cells were then used to evaluate their relative ATP content by luminescence using the Varioskan™ LUX plate reader, 24 hours after plating. For these experiments, which required larger numbers of cells, a cut-off of 10% was used to define the ATP-high and ATP-low cell populations. Data represent the mean fold increase±SD over ATP-low 10% cells, n=3. Unpaired t-test, ****p<0.0001.The observed increases in ATP levels were reduced to 3-fold by 24 hours after plating the ATP-high cells as a 2D monolayer, indicating that the highly energetic, ATP-high phenotype is relatively transient, consistent with a more stem-like phenotype.

FIGS. 10B through 10E show the results of metabolic flux analysis on the ATP-high and ATP-low sub-populations. The OCR (oxygen-consumption rate) was determined using the Seahorse XFe96, via metabolic flux analysis. Note that the ATP-high MCF7 cell population shows an increase in both basal and maximal respiration, as well as mitochondrial ATP-production. Cell populations were analyzed 24 hours after plating. Data represent the % fold increase±SD over ATP-low 10% cells, n=3. Unpaired t-test, * p<0.05, ** p<0.005. The ECAR (extracellular acidification rate) was determined using the Seahorse XFe96, via metabolic flux analysis. Note that the ATP-high MCF7 cell population shows an increase in glycolysis. Cell populations were analyzed 24 hours after plating. Data represent the % average fold increase±SD over ATP-low 10% cells, n=3. Unpaired t-test, ns=not significant, *** p<0.0005. The energetic profiles in FIGS. 10B and 10C show that the ATP-high sub-population is metabolically active relative to the ATP-low population. Following 24 hours after cell attachment, ATP-high MCF7 cell monolayers showed a 2-fold increase in basal respiration, a 1.5-fold increase in maximal respiration and a 3-fold increase in ATP production. Similarly, ATP-high MCF7 monolayer cells also showed a 1.5-fold increase in basal glycolytic rate. The glycolytic rates in FIGS. 10D and 10E demonstrate that the ATP-high sub-population is significantly more bioenergetic than the ATP-low sub-population.

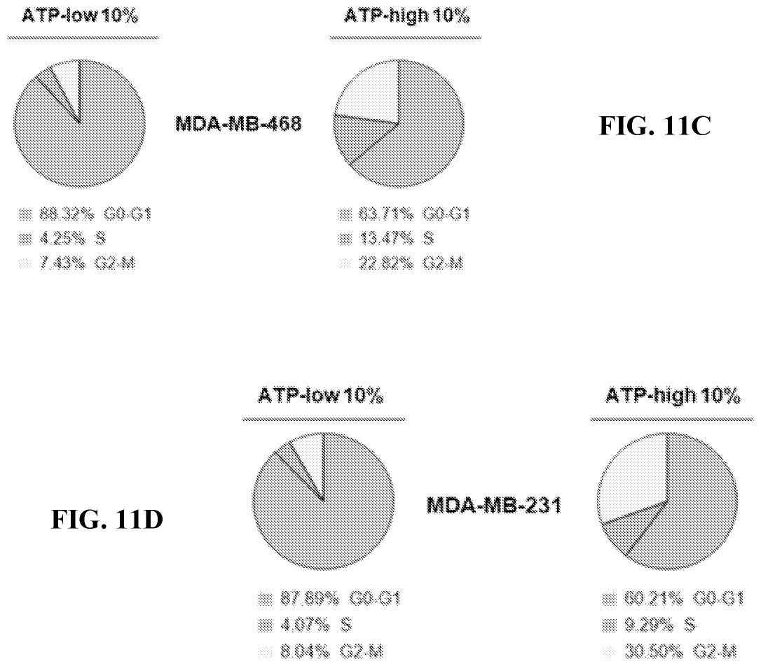

An evaluation of the proliferative capacity of the ATP-high cell sub-population reveals that the ATP-high cell sub-populations are strikingly more proliferative than the ATP-low sub-populations, in a variety of cancer types. FIGS. 11A-11D show cell cycle progression in MCF7, T47D, MDA-MB-468, and MDA-MB-231 cells, using FACS analysis with propidium iodide to detect DNA-content. As can be seen, the ATP-high cell sub-populations were strikingly more proliferative than the ATP-low, with a shift from the G0/G1-phase to the S-phase and the G2/M-phase. More specifically, the G0/G1-phase was reduced from approximately 80-88% to 60-64%, while the S-phase was increased from 4-8% to 9-21%. Similarly, the G2/M-phase was increased, from 7-12% to 17-30%. These clear increases in cell cycle progression in the ATP-high cell sub-population, relative to the ATP-low sub-population, were observed in all 4 cell lines. Overall, this represents a 1.9- to 3.6-fold increase in the number of cells in S-phase and a 1.8- to 3.8-fold increase in the number of cells in the G2/M-phase, across the 4 cell lines tested. Interestingly, the largest increase in S-phase was observed in MCF7 cells, while the largest increase in the G2/M-phase was observed in MDA-MB-231 cells.

Conversely, the ATP-low population in each cell line was essentially quiescent, with 80-88% of the cells in the G0/G1-phase of the cell cycle, demonstrating a predominant phenotype of cell cycle arrest. Thus, the ATP-low cell sub-population fits well with the current definition of cancer cell dormancy.

Therefore, high ATP levels are a primary determinant of “stemness” traits, anchorage independent growth, and cell proliferation. As such, the practical approach described herein allows for successfully isolating the bioenergetically “fittest” and most proliferative cancer cells, from the total cell population, and forming a new composition of cells having unique phenotypic properties. These properties have implications for drug-resistance.

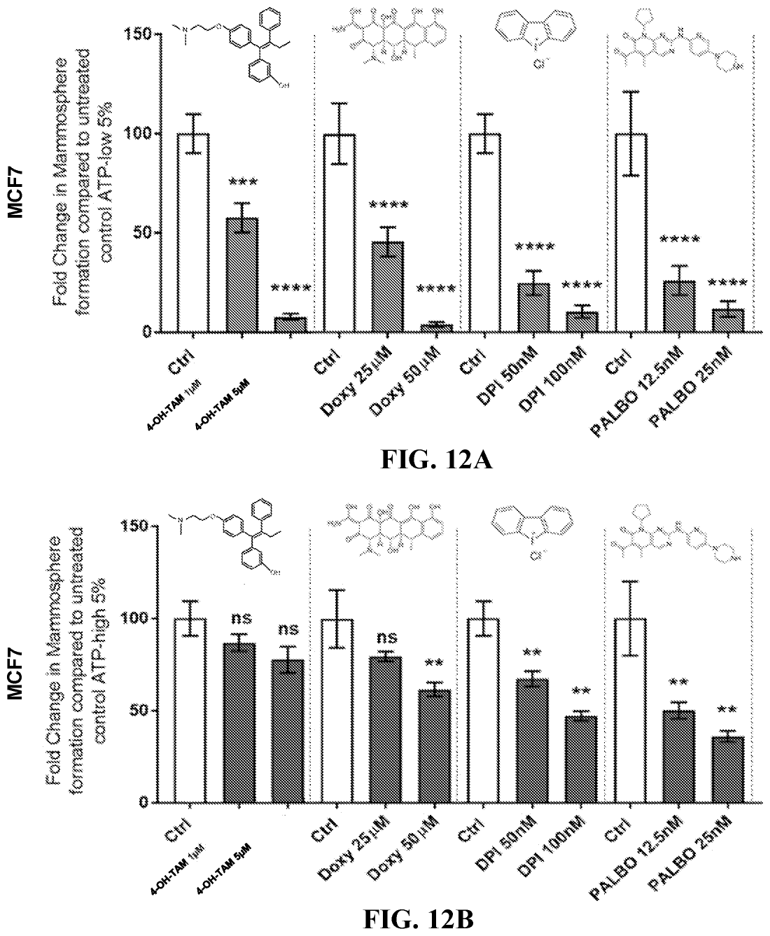

MCF7 cells in the ATP-high subpopulation show a multi-drug resistance phenotype. The 3D-mammosphere assay was used to explore the differential sensitivity of ATP-high and ATP-low MCF7 cell sub-populations to four different classes of drugs, using as a functional readout of drug-resistance. The drug classes include Tamoxifen, doxycycline, DPI, and Palbociclib. FIG. 12A shows results for the ATP-low sub-population (bottom 5%), and FIG. 12B shows results for the ATP-high subpopulation (top 5%). Two concentrations for each drug class are shown in FIGS. 12A and 12B.

Tamoxifen is an FDA-approved drug routinely used to clinically target ER(+) breast cancer cells, that often leads to Tamoxifen-resistance and treatment failure, resulting in tumor recurrence and distant metastasis. Interestingly, 3D-mammosphere formation by ATP-low MCF7 cells was remarkably sensitive to Tamoxifen treatment, resulting in a reduction by ˜40% at 1μM and by >90% at 5 μM. In contrast, FIG. 12B shows that 3D-mammosphere formation by ATP-high MCF7 cells was strikingly resistant to Tamoxifen, as 3D-mammosphere formation remained high at 5 μM, representing >80% of the vehicle-treated control levels. Thus, ATP-high MCF7 cells are clearly Tamoxifen-resistant.

FIG. 12B shows that ATP-high MCF7 cells were also resistant to a mitochondrial OXPHOS inhibitor, namely diphenyleneiodonium (DPI). For example, DPI treatment of ATP-low cells reduced 3D-mammosphereformation by >90% at 100 nM. On the other hand, DPI treatment (100 nM) of ATP-high cells only reduced 3D-mammosphere formation by ˜55%. Therefore, both sub-populations were sensitive to a mitochondrial inhibitor, but ATP-high cells were clearly more resistant.

Doxycycline is an FDA-approved antibiotic which behaves as an inhibitor of mitochondrial ribosome translation. Comparing FIG. 12A to FIG. 12B shows that the ATP-high sub-population was largely resistant to Doxycycline, at concentrations that were highly effective in ATP-low MCF7 cells, namely 25 μM and 50 μM.

The efficacy of Palbociclib, an FDA-approved CDK4/6 inhibitor, is also evident in FIGS. 12A and 12B. Palbociclib treatment of ATP-low cells reduced 3D-mammosphere formation by ˜75% at 12.5 nM. However, Palbociclib treatment (12.5 nM) of ATP-high cells only reduced 3D spheroid formation by -50%. As such, ATP-high cells were also more resistant to a CDK4/6 inhibitor. As can be seen, the ATP-high sub-population is a phenotype having resistance to several drug types.

Biotracker-ATP-Red 1 was compared with well-established markers of stemness, CD44, and ALDH-activity. In order to directly compare the effectiveness of ATP-Red 1 with other CSC markers, a double-labeling strategy was applied to both MCF7 and MDA-MB-231 cells. The cells were double-labeled for CD44 and ATP, using different fluorescent channels for detection. In the case of CD44 and ATP, this resulted in 4 experimental groups: CD44-high/ATP-high, CD44-high/ATP-low, CD44-low/ATP-high, and CD44-low/ATP-low.

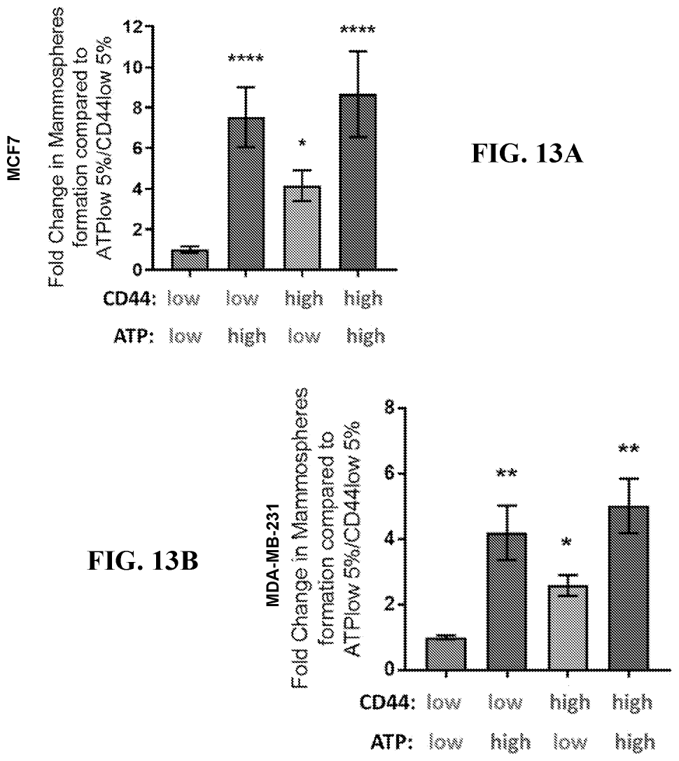

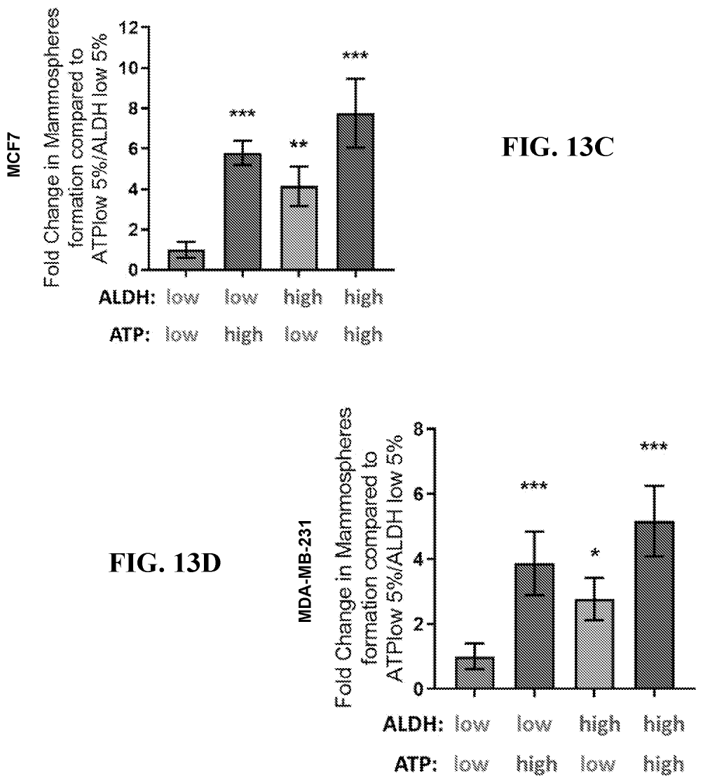

After cell sorting, the resulting four sub-populations were then subjected to the 3D-mammosphere assays, as a functional read-out of stemness. ATP versus CD44 cell surface expression. 3D anchorage-independent growth was measured in the different cell sub-populations, as a functional readout of stemness, using both MCF7 and MDA-MB-231 lines, after cell sorting. Briefly, 2D-monolayers were first co-stained with both BioTracker-ATP (PE channel) and Anti-CD44 (APC-channel) and subjected to flow cytometry, using the SONY SH800 cell sorter. After cell counting, 5×10³ cells were seeded in poly-HEMA coated 6-well plates and 3D-mammospheres were counted 5 days after plating. As shown in FIGS. 13A and 13B, CD44-low/ATP-low cells showed the least anchorage-independent growth, as expected given the phenotypic properties of these sub-populations. Therefore, CD44-low/ATP-low cells were chosen as the point for normalization. Two cell sub-populations showed the most anchorage independent growth: CD44-high/ATP-high and CD44-low/ATP-high. Therefore, high levels of ATP are the dominant determinant of stemness, as compared with CD44, in both MCF7 and MDA-MB-231 cells.

Considering only the CD44-high population and double-labeling with ATP allowed for separating the CD44-high population into 2 sub-populations, one with high capacity for propagation (CD44-high/ATP-high) and one with low capacity for propagation (CD44-high/ATP-low). Therefore, the CD44-high/ATP-low population clearly showed significantly less anchorage-independent growth and represents a more “dormant” sub-population of CD44(+) CSCs.

Virtually identical results were also obtained by double-labeling with ADLH-activity and ATP. 3D anchorage-independent growth was measured in the different cell sub-populations, as a functional readout of stemness, using both MCF7 and MDA-MB-231 lines, after cell sorting. Briefly, 2D monolayers were first co-stained with both BioTracker-ATP (PE channel) and for ALDH-activity (APC-channel) and subjected to flow cytometry, using the SONY SH800 cell sorter. After cell counting, 5×10³ cells were seeded in poly-HEMA coated 6-well plates and 3D mammospheres were counted 5 days after plating. FIGS. 13C and 13D show results of the mammosphere formation assay for MCF7 and MDA-MB-231 cell lines, respectively, double-labeled for ALDH-activity and ATP. As expected, the two cell populations that showed the most anchorage-independent growth were ALDH-high/ATP-high and ALDH-low/ATP-high. Therefore, high levels of ATP are the dominant determinant of stemness, as compared with ALDH, in both MCF7 and MDA-MB-231 cells. Similarly, double-labeling with ATP allows for the separation of the ALDH-high population into 2 sub-populations, one with high capacity for propagation (ALDH-high/ATP-high) and one with low capacity for propagation (ALDH-high/ATP-low).

The foregoing demonstrates that the present approach is a powerful and effective approach for sub-fractionating CSCs into a more active, hyper-proliferative sub-population, and a more dormant sub-population, using ATP as a secondary marker for dormancy. The results also indicate that ATP levels are a functional regulator of dormancy in CSCs.

The role of mitochondrial ATP in cell migration, invasion and spontaneous metastasis was also explored. The data demonstrate that mitochondrial ATP is an energetic biomarker for the process of cancer cell metastasis. MDA-MB-231 cells are a well-established model for the study of cell motility and metastasis, both in vitro and in vivo. The ability of ATP-high and ATP-low subpopulations of MDA-MB-231 cells to undergo cell migration and invasion were evaluated by employing a modified Boyden chamber assay, using Transwells. The bulk (5%) population was also selected for comparison purposes. To study invasion, the Transwells were coated with extracellular matrix, namely Matrigel, to prevent simple cell migration. For both cell migration and invasion assays, serum was used as a chemoattractant. Migration and invasion parameters were independently quantitated, using both crystal violet staining intensity and cell number.

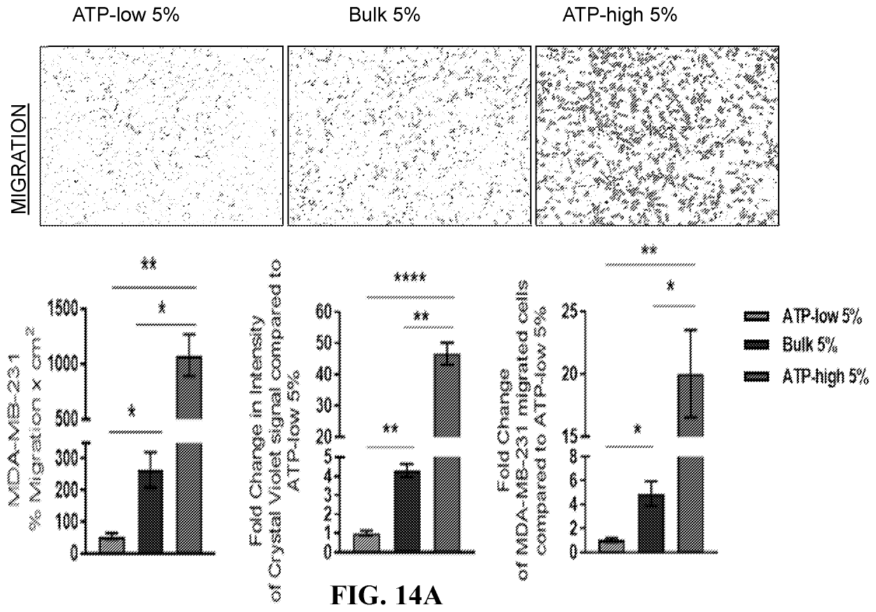

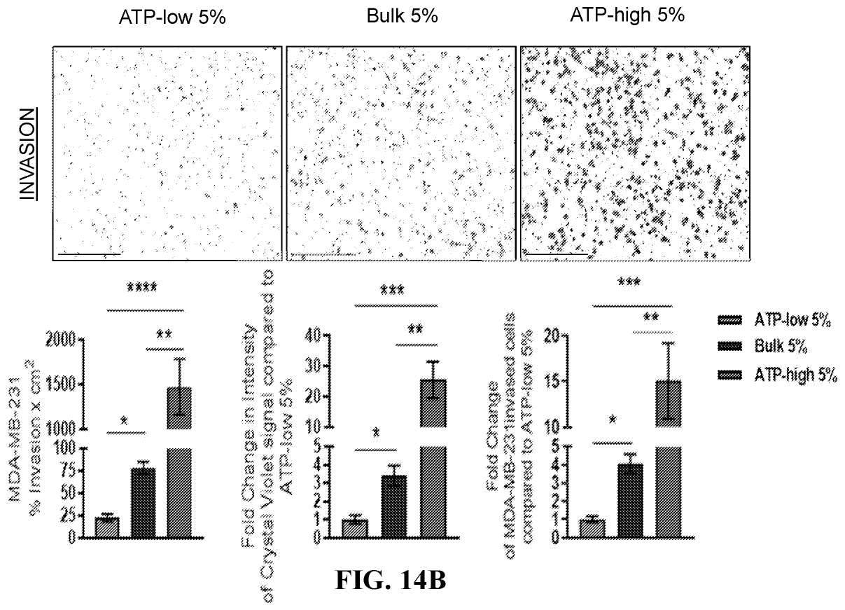

FIGS. 14A and 14B show the results of this migration and invasion analysis. The ATP-high MDA-MB-231 cells showed a 20- to 40-fold increase in their ability to undergo cell migration, relative to ATP-low cells. As expected bulk (5%) cells showed an intermediate phenotype. ATP-high MDA-MB-231 cells showed a 15- to 25-fold increase in their ability to undergo invasion, relative to ATP-low cell population. As such, ATP-high MDA-MB-231 cells represent the pro-metastatic cell sub-population in vivo.

For further evaluation, a well-established in vivo metastasis assay, involving the chorio-allantoic membrane (CAM) in chicken eggs, was used to quantitatively measure spontaneous metastasis. After cell sorting to isolate ATP-high and ATP-low cell sub-populations, an inoculum of 30,000 cells (MDA-MB-231) was added onto the CAM of each egg (day E9) and then eggs were randomized into groups. On day E18, the lower CAM was collected to evaluate the number of metastatic cells, as analyzed by qPCR with specific primers for Human Alu sequences. Non-injected eggs were also evaluated in parallel, as a negative control for specificity. Greater than 20 eggs were processed for each experimental condition.

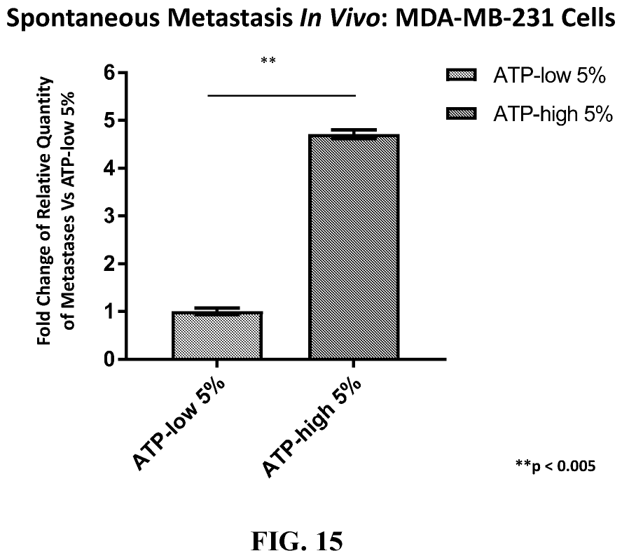

FIG. 15 shows the results of the spontaneous metastasis in vivo CAM assay. The data illustrate that MDA-MB-231 cells in the ATP-high sub-population were 4.5-fold more metastatic than ATP-low cell sub-population. These sub-populations were derived from the same cell line. Therefore, MDA-MB-231 cells in the ATP-high sub-population represent the pro-metastatic CSC sub-population. As discussed above in connection with FIG. 4C, MDA-MB-231 cells in the ATP-high sub-population also over-express two CTC and metastasis markers (VCAM-1 and Ep-CAM), indicating that the hyper-proliferative CSCs are the CTCs responsible for seeding distant metastasis.

The present approach can therefore be used to detect the potential of a cancer to metastasize. For example, a biological sample from a cancer may be metabolically fractionated to assess the content of the ATP-high sub-population, and that content may be used to estimate the likelihood of the cancer to metastasize. Early detection and analysis of cells in a cancer patient's ATP-high sub-population provides invaluable opportunities to diagnose the risk of metastasis and identify an appropriate treatment, such as with an ATP-depletion therapeutic as discussed herein.

The Tempo-ATP protein-biosensor to purify ATP-high MCF7 cells provides independent validation of the use of ATP as a new biomarker for stemness in cancer cells. Tempo-ATP, a fluorescent protein-biosensor, is a completely different probe for detecting ATP levels in living cells, and was used for detecting high and low levels of ATP.

Tempo-ATP-MCF7 cells, recombinantly over-expressing a cytosolic fluorescent protein ATP-biosensor, were custom-generated by Tempo-Bioscience, Inc. (San Francisco, Calif., USA), using a puromycin-resistance marker for cell selection. This protein-based fluorescent ATP-biosensor has an excitation wavelength of 517-519-nm and an emission of 535-nm. It consists of an ATP-binding peptide, fused in-frame with a GFP-like fluorescent reporter protein. The Tempo-ATP-MCF7 cells were sorted for GFP content, as a surrogate marker for cytoplasmic ATP-content, using a flow cytometer (Excitation=517-519-nm; Emission=535-nm). After cell counting, equal numbers of single cells were then used to evaluate their metabolic and phenotypic behavior.

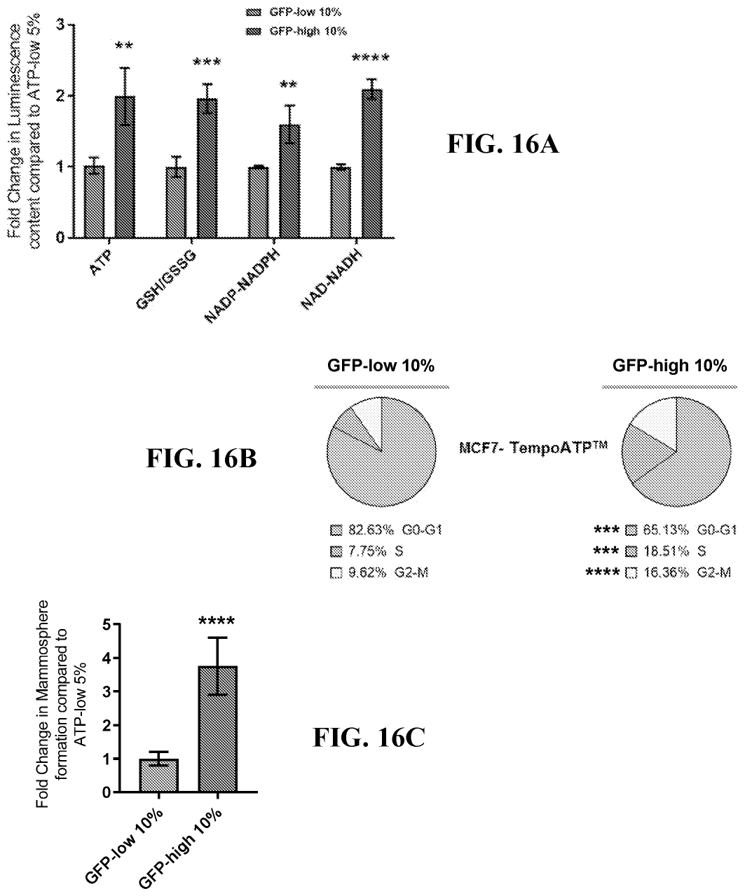

The results are shown in FIGS. 16A-16C. The relative increase in luminescence of the GFP-high sub-population, relative to the GFP-low sub-population, is shown in FIG. 16A. Cell cycle progression data are summarized in FIG. 16B, and mammosphere formation assay results are shown in FIG. 16C. As expected based on the discussion thus far, the ATP-high Tempo-MCF7 cells showed significant increases in ATP, more reduced glutathione, NADP/H and NAD/H, as well as increases in cell cycle progression and 3D anchorage-independent growth. The Tempo-ATP data independently validation the BioTracker ATP-Red 1 results, and specifically, that high-ATP levels are a key determinant of anti-oxidant capacity, cell proliferation, and 3D anchorage-independent growth. Although Tempo-ATP was effective, BioTracker ATP-Red 1 was significantly more effective, because of its direct localization within mitochondria, the main cellular source ATP production.

The present approach demonstrates that high ATP production is a key driver of “stemness” traits and proliferation in cancer cells. The observations disclosed herein could explain the molecular basis of metabolic heterogeneity observed in the cancer cell population, as well as its relationship to phenotypic behaviors, such as i) rapid cell cycle progression and ii) anchorage-independent growth, which are both required for the metastatic dissemination of CSCs in vivo.

As demonstrated, ATP may be used as a biomarker to metabolically fractionate a cancer cell population, and identify hyper-prolific and dormant sub-populations. This, in turn, indicates that ATP-depletion therapy may be effective for treating the hyper-prolific sub-populations, and reduce or eliminate the likelihood of tumor recurrence and metastasis.

Under the present approach, a vital fluorescent dye that allows one to measure ATP levels in living cells, such as BioTracker ATP-Red 1, may be used as an imaging probe for metabolic fractionation. More specifically, BioTracker ATP-Red 1 staining may be coupled with a bioenergetic fractionation scheme, in which the total cell population is subjected to flow cytometry, to isolate the ATP-high and ATP-low sub-populations of the population. MCF7 cells, an ER(+) human breast cancer cell line, were used in many of the examples discussed above, but it should be appreciated that the present approach may be used for any cell line, and any cancer type. The metabolic fractionation approach allows for isolating the most “energetic” cancer cells within the total cell population. Advantageously, the resulting ATP-high cancer cell sub-population may be targeted for eradication via ATP-depletion therapy, and serve as a basis for drug discovery and development. Given the phenotypic properties, the ATP-high sub-population may also be used for evaluating therapies to prevent or reduce the likelihood of recurrence and metastasis.

In a parallel line of research, the inventors identified over 20 mitochondrially-targeted therapeutics that could be used to effectively achieve ATP-depletion therapy. These potential therapeutics include: FDA-approved drugs (Doxycycline, Tigecycline, Azithromycin, Pyrvinium pamoate, Atovaquone, Bedaquiline, Niclosamide, Irinotecan); natural products/nutraceuticals (Actinonin, CAPE, Berberine, Brutieridin, Melitidin); and experimental compounds (Oligomycin, AR-C155858, Mitoriboscins (see International Application No. PCT/US2018/022403, filed Mar. 14, 2018, and incorporated by reference in its entirety.), Mitoketoscins (see International Application PCT/US2018/039354, filed Jun. 25, 2018, and incorporated by reference in its entirety), Mitoflavoscins (see International Patent Application PCT/US2018/057093, filed Oct. 23, 2018 and incorporated by reference in its entirety.), TPP derivatives (including Dodecyl-TPP and 2-Butene-1,4-bis-TPP, see International Patent Application PCT/US2018/062174, filed Nov. 21, 2018 and incorporated by reference in its entirety.)). A triple-combination of two antibiotics together with Vitamin C (Doxycycline, Azithromycin and Ascorbic acid) was found to be particularly potent for targeting mitochondria, inducing ATP-depletion and inhibiting CSC propagation, at sub-antimicrobial levels (see International Patent Application PCT/US2019/066541, filed Dec. 16, 2019 and incorporated by reference in its entirety). The ATP-depletion compound may be an existing compound modified to increase efficacy, cell membrane penetration, and/or mitochondrial uptake, such as those described in International Patent Application PCT/US2018/033466, filed May 18, 2018 and incorporated by reference in its entirety, and International Patent Application PCT/US2018/062956, filed Nov. 29, 2018 and incorporated by reference in its entirety. For example, Doxycycline conjugated with a fatty acid, such as Myristate, may be used as an ATP-depletion compound. In some instances, it may be appropriate to administer an increased dose of a compound, such as when the ATP-high sub-population shows resistance to the compound at a dose normally prescribed in the art. A compound may be administered in It should be appreciated that any of the foregoing compounds may be used as an ATP-depletion therapeutic, to target the ATP-high sub-population, and prevent or reduce the likelihood of recurrence and metastasis. It should also be appreciated that any of the foregoing compounds may be used as a therapeutic agent to be administered to a cancer patient when the expression levels of the 5-member gene signature of ABCA2, ATP5F1C, COX20, NDUFA2, and UQCRB, in a biologic sample of the patient's cancer, are found to be elevated relative to expression levels in a non-cancerous biologic sample from the patient.

As many of the compounds are repurposed FDA-approved antibiotics, with excellent safety profiles, Phase II clinical trials are warranted. For example, a Phase II clinical pilot study of Doxycycline has already shown that this over 50-year-old antibiotic is indeed effective in metabolically targeting the CSC population in early breast cancer patients, as demonstrated using CD44 and ALDH1 as specific CSC markers. Mitochondrial ATP-depletion therapy is expected to functionally mimic fasting and/or caloric restriction, thereby more effectively starving CSCs to death. Under the present approach, fasting and/or caloric restriction may be included as part of anti-cancer therapy, to increase the effectiveness of an ATP-depletion therapy. For example, a patient receiving ATP-depletion therapy may fast for a period such as 12, 16, 24, 36, or 48 hours, before receiving administration of a therapeutic compound, and/or may fast for a period such as 12, 16, 24, 36, or 48 hours, after receiving administration of the therapeutic compound. In some embodiments, the fast may take place before and after administration of the therapeutic compound, to increase the ATP-depletion effect. This has important implications for cancer prevention and for potentially extending human lifespan during aging.

Cells in the ATP-high sub-populations show a multi-drug resistant phenotype, with enhanced anti-oxidant capacity. Previous studies have shown that high anti-oxidant capacity, due to increased levels of reduced glutathione, elevated NADPH, and activated NRF2 signaling, significantly contributes to the onset of multi-drug resistance. MCF7 cells in the ATP-high sub-population have an increased anti-oxidant capacity, with elevated levels of reduced glutathione, and are intrinsically resistant to four different classes of drugs (Tamoxifen, Palbociclib, Doxycycline and DPI). Therefore, the existence of the ATP-high CSC phenotype may help to mechanistically explain the pathogenesis of multi-drug resistance, during cancer therapy. In this context, current cancer therapy may allow only the metabolically “fittest” cancer cells to survive. Those cells, in turn, present the greatest risk of recurrence and metastasis.

The data disclosed above also show a direct causal relationship between mitochondrial “power” and Tamoxifen-resistance. For example, MCF7-TAMR cells that were generated via chronic exposure to increasing concentrations of Tamoxifen, resulting in Tamoxifen-resistance, showed elevated levels of mitochondrial OXPHOS and ATP production. In MCF7-TAMR cells, acquired Tamoxifen-resistance was due to the over-expression of two key anti-oxidant proteins (NQO1 and GCLC) and their positive metabolic effects on mitochondrial metabolism, as revealed by unbiased proteomics analysis. In addition, recombinant over-expression of either NQO1 or GCLC in MCF7 cells autonomously conferred about a 2-fold increase in mitochondrial ATP-production and Tamoxifen-resistance. Moreover, recombinant over-expression of a somatic mutation (Y537S) in the estrogen receptor (ER-alpha; ESR1), clinically associated with acquired Tamoxifen-resistance in breast cancer patients, genetically conferred elevated mitochondrial biogenesis, OXPHOS and high ATP production. The proteomic profiles of MCF7-TAMR cells and MCF7-ESR1(Y537S) cells also showed considerable overlap in the biological processes that were functionally activated. Finally, 60 gene products functionally-associated with mitochondrial ATP production, were predictive of Tamoxifen-resistance in ER(+)/Luminal A breast cancer patients. These predictive biomarkers included 18 different mitochondrial ribosomal proteins (MRPs) and over 20 distinct components of the mitochondrial OXPHOS complexes. The data disclosed herein show that “naïve” MCF7 cells in the ATP-high sub-population are intrinsically resistant to Tamoxifen, without any prior exposure to Tamoxifen. This has important clinical implications for optimizing the effectiveness of hormonal breast cancer therapy.

It has been previously reported that treatment with conventional chemotherapeutic regimens actually increases the number of CSCs, while selectively killing “bulk” cancer cells. Until this disclosure, no metabolic hypotheses have been proposed to explain the phenomenon. Chan and colleagues (from Genentech, Inc.) examined the effects of gemcitabine and etoposide on the total cancer cell population. Remarkably, they observed that after treatment with gemcitabine and etoposide, the population of surviving cells showed an increase in ATP content, elevated mitochondrial mass, with more mitochondrial respiration. However, they did not propose a mechanistic explanation for these observations, nor did they consider the CSC population. Instead, they simply concluded that measuring ATP is not a good read-out to assess the effectiveness of chemo-therapeutic agents. Given the data disclosed herein, an alternate interpretation of their results is that gemcitabine and etoposide selectively killed the ATP-low and bulk sub-populations of cancer cells, thereby enriching the “energetic” ATP-high sub-population, which are more stem-like and drug-resistant. Therefore, new drug discovery should be initiated to help eradicate the ATP-high sub-population of cancer cells.

Higher intracellular ATP levels have also been suggested to account for acquired drug-resistance to oxaliplatin and cisplatin, in a variety of chronically-treated colon and ovarian cancer cell lines (HT29, HCT116, A2780), although a diverse number of mechanisms have been proposed, including increased glycolysis and/or mitochondrial metabolism. However, in previous studies, ATP levels were measured only after chronically selecting for the drug resistant cell population. Therefore, a direct cause-effect relationship between ATP production and drug resistance could not be established.

Previously, the inventors used a more indirect method to isolate “energetic” cancer stem cells (e-CSCS), which employed auto-fluorescence to detect intracellular FAD, FMN and riboflavin content. See International Patent Application PCT/US2019/037860, filed Jun. 19, 2019, which is incorporated by reference in its entirety. However, the use of ATP-Red 1 is a direct method and is a substantial improvement. For example, the use of high auto-fluorescence (AF; top 5%) to fractionate MCF7 2D monolayers resulted in an AF-high population of cells, with a 1.5-fold increase in anchorage independent growth and a near 2-fold increase in ATP production. In contrast, in the present approach, the use of ATP-Red 1 (top 5%) resulted in a sub-population of ATP-high MCF7 monolayer cells having a 9-fold increase in anchorage independent growth and over a 15-fold increase in ATP content. The ATP-high sub-populations from other cancer cell lines (T47D, MDA-MB-231 and MDA-MB-468) showed similarly hyper-proliferative characteristics. Therefore, the use of ATP as a direct energetic biomarker is far superior to auto-fluorescence. In addition, ATP-Red 1 was also effective for metabolically fractionating the three other breast cancer cell lines tested.

According to the conventional view of tumor dormancy, dormant cancer cells undergo slower rates of cell proliferation and/or cell cycle arrest (quiescence), to avoid therapy-induced cell death, leading to multi-drug resistance. The data disclosed herein show just the opposite: MCF7 cells in the ATP-low sub-population were less proliferative, with over 87% of the cells in the GO/G1 phase of the cell cycle, but were more sensitive to 4 different classes of drugs, using the 3D-mammosphere assay as a readout. Conversely, MCF7 cells in the ATP-high sub-population were significantly more proliferative, with over 38% of the cells in either S-phase or G2/M, showing a clear multi-drug resistance phenotype. Therefore, high levels of mitochondrial ATP are a key driver of both cell proliferation and drug-resistance, as they represent the energetically “fittest” population of cancer cells.

The inventors have shown that treatments with a panel of distinct anti-mitochondrial therapeutics i) metabolically induce ATP-depletion and ii) are sufficient to potently inhibit cancer cell metastasis, using an in vivo xenograft animal model. These results indicate that high ATP levels are critical for the processes of CSC metastasis, and are consistent with the data disclosed herein, showing that that ATP-high CSCs are hyper-proliferative, stem-like, anchorage-independent, with increases in anti-oxidant capacity and intrinsic multi-drug resistance. Therefore, the ATP-high CSC that may be isolated using the present approach is likely responsible for tumor recurrence and metastasis in vivo.

The bioinformatic analysis described above shows that ATP-related genes are closely associated with stemness, proliferation and metastasis, especially ATP5F1C, which encodes the gamma-subunit of the catalytic core of the mitochondrial ATP synthase. Moreover, ATP5F1C is a prognostic biomarker of tumor recurrence and distant metastasis, as well as a marker of treatment failure in ER(+) patients undergoing Tamoxifen therapy. Also, ATP-high MDA-MB-231 cells showed dramatic increases in their capacity to undergo both cell migration and invasion in vitro, as well as spontaneous metastasis in vivo. Mitochondrial ATP, then, plays a critical role in metastatic dissemination. As such, inhibitors of mitochondrial ATP synthesis should be effective as potential therapeutics for conveying metastasis prophylaxis, for eradicating the CSCs in the ATP-high sub-population.

Pharmaceutical compositions of the present approach include an ATP-depleting compound (as identified above) in any pharmaceutically acceptable carrier. If a solution is desired, water may be the carrier of choice for water-soluble compounds or salts. With respect to water solubility, organic vehicles, such as glycerol, propylene glycol, polyethylene glycol, or mixtures thereof, can be suitable. Additionally, methods of increasing water solubility may be used without departing from the present approach. In the latter instance, the organic vehicle can contain a substantial amount of water. The solution in either instance can then be sterilized in a suitable manner known to those in the art, and for illustration by filtration through a 0.22-micron filter. Subsequent to sterilization, the solution can be dispensed into appropriate receptacles, such as depyrogenated glass vials. The dispensing is optionally done by an aseptic method. Sterilized closures can then be placed on the vials and, if desired, the vial contents can be lyophilized. Embodiments including a second inhibitor compound, such as a glycolysis inhibitor or an OXPHOS inhibitor, may co-administer a form of the second inhibitor available in the art. The present approach is not intended to be limited to a particular form of administration, unless otherwise stated.

In addition to the ATP-depleting compound, pharmaceutical formulations of the present approach can contain other additives known in the art. For example, some embodiments may include pH-adjusting agents, such as acids (e.g., hydrochloric acid), and bases or buffers (e.g., sodium acetate, sodium borate, sodium citrate, sodium gluconate, sodium lactate, and sodium phosphate). Some embodiments may include antimicrobial preservatives, such as methylparaben, propylparaben, and benzyl alcohol. An antimicrobial preservative is often included when the formulation is placed in a vial designed for multi-dose use. The pharmaceutical formulations described herein can be lyophilized using techniques well known in the art.

In embodiments involving oral administration of an ATP-depleting compound, the pharmaceutical composition can take the form of capsules, tablets, pills, powders, solutions, suspensions, and the like. Tablets containing various excipients such as sodium citrate, calcium carbonate and calcium phosphate may be employed along with various disintegrants such as starch (e.g., potato or tapioca starch) and certain complex silicates, together with binding agents such as polyvinylpyrrolidone, sucrose, gelatin and acacia. Additionally, lubricating agents such as magnesium stearate, sodium lauryl sulfate, and talc may be included for tableting purposes. Solid compositions of a similar type may be employed as fillers in soft and hard-filled gelatin capsules. Materials in this connection also include lactose or milk sugar, as well as high molecular weight polyethylene glycols. When aqueous suspensions and/or elixirs are desired for oral administration, the compounds of the presently disclosed subject matter can be combined with various sweetening agents, flavoring agents, coloring agents, emulsifying agents and/or suspending agents, as well as such diluents as water, ethanol, propylene glycol, glycerin and various like combinations thereof. In embodiments having a carbocyanine compound with a second inhibitor compound, the second inhibitor compound may be administered in a separate form, without limitation to the form of the carbocyanine compound.

Additional embodiments provided herein include liposomal formulations of an ATP-depleting compound disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound can be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques. The liposomal formulations comprising the active compounds disclosed herein can be lyophilized to produce a lyophilizate, which can be reconstituted with a pharmaceutically acceptable carrier, such as water, to regenerate a liposomal suspension.

With respect to pharmaceutical compositions, the pharmaceutically effective amount of an ATP-depleting compound herein will be determined by the health care practitioner, and will depend on the condition, size and age of the patient, as well as the route of delivery. In one non-limited embodiment, a dosage from about 0.1 to about 200 mg/kg has therapeutic efficacy, wherein the weight ratio is the weight of the ATP-depleting compound, including the cases where a salt is employed, to the weight of the subject. In some embodiments, the dosage can be the amount of compound needed to provide a serum concentration of the active compound of up to between about 1 and 5, 10, 20, 30, or 40 μM. In some embodiments, a dosage from about 1 mg/kg to about 10, and in some embodiments about 10 mg/kg to about 50 mg/kg, can be employed for oral administration. Typically, a dosage from about 0.5 mg/kg to 5 mg/kg can be employed for intramuscular injection. In some embodiments, dosages can be from about 1 μmol/kg to about 50 μmol/kg, or, optionally, between about 22 μmol/kg and about 33 μmol/kg of the compound for intravenous or oral administration. An oral dosage form can include any appropriate amount of active material, including for example from 5 mg to, 50, 100, 200, or 500 mg per tablet or other solid dosage form.

The following paragraphs describe the materials and methods used in connection with the data and embodiments set forth herein. It should be appreciated that those having an ordinary level of skill in the art may use alternative materials and methods generally accepted in the art, without deviating from the present approach.

Cell lines and Reagents: ER(+) [MCF7 and T47D] and triple-negative [MDA-MB-231 and MDA-MB-468] human breast cancer cell lines were purchased from the American Type Culture Collection (ATCC). ATP-Red 1 (also known as BioTracker™ ATP-Red Live Cell Dye; #SCT045) was obtained commercially from Sigma-Aldrich, Inc.

The HeatMap of FIG. 1A was prepared using the GSE36953 GEO DataSet, previously deposited in the NCBI database. Total RNA was prepared from MDA-MB-231 cells, a TNBC cell line, under three different growth conditions: 2D-adherent growth, 3D-anchorage-independent growth and in vivo tumor growth. Analysis was performed with the Affymetrix Human Genome U133 Plus 2.0 Array. The HeatMap was generated with QIAGEN OmicSoft Suite Software. ATP-related genes were transcriptionally upregulated under both 3D growth conditions (anchorage-independent and in vivo tumors), all relative to 2D-adherent growth

Flow Cytometry after Vital Staining with ATP-Red 1: Human breast cancer cell lines were first grown either as a 2D-monolayer or as 3D-spheroids. Then the cells were collected and dissociated into a single-cell suspension, prior to analysis or sorting by flow-cytometry with a SONY SH800 Cell Sorter. Briefly, ATP-high and ATP-low sub-populations of cells were isolated after vital staining with the probe ATP-Red 1. The ATP-high and ATP-low cell sub-populations were selected by gating, within the ATP-Red 1 signal. Unless otherwise stated, cells with the lowest (bottom 5% or 10%) fluorescent signal, or the highest (top 5% or 10%) fluorescent signal, were collected as ATP-low and ATP-high, respectively. The cells outside the gates were discarded during sorting, due to the gate settings. However, such settings are often required to ensure high-purity during sorting. Data were analyzed with FlowJo 10.1 software.

ATP assay with Cell-Titer-Glo: Cell-Titer-Glo (#G7570) was obtained from Promega, Inc., and was used according to the manufacturer's recommendations, to measure ATP levels in lysed cells. Cell-Titer-Glo is a luciferase-based assay system.

3D Anchorage Independent Growth Assay: A single cell suspension was prepared using enzymatic (lx Trypsin-EDTA, Sigma Aldrich, cat. #T3924), and manual disaggregation (25 gauge needle). Five thousand cells were plated with in mammosphere medium (DMEM-F12/B27/20ng/m1 EGF/PenStrep), under non-adherent conditions, in six wells plates coated with 2-hydroxyethylmethacrylate (poly-HEMA, Sigma, cat. #P3932). Cells were grown for 5 days and maintained in a humidified incubator at 37° C. at an atmospheric pressure in 5% (v/v) carbon dioxide/air. After 5 days, 3D spheroids with a diameter greater than 50 μm were counted using a microscope, fitted with a graticule eye-piece, and the percentage of cells which formed spheroids was calculated and normalized to one (1=100% MFE; mammosphere forming efficiency). Mammosphere assays were performed in triplicate and repeated three times independently.

Metabolic Flux Analysis: Extracellular acidification rates and oxygen consumption rates were analyzed using the Seahorse XFe96 analyzer (Agilent/Seahorse Bioscience, USA). Cells were maintained in DMEM supplemented with 10% FBS (fetal bovine serum), 2 mM GlutaMAX, and 1% Pen- Strep. Twenty-thousand breast cancer cells were seeded per well, into XFe96-well cell culture plates, and incubated at 37° C. in a 5% CO2 humidified atmosphere for at least 12 hours to allow cell attachment. After about 24 hours, MCF7 cells were washed in pre-warmed XF assay media, or for OCR measurement, XF assay media supplemented with 10 mM glucose, 1 mM Pyruvate, 2 mM L-glutamine, and adjusted at 7.4 pH. Cells were then maintained in 175 μL/well of XF assay media at 37° C., in a non-CO2 incubator for 1 hour. During the incubation time, 25 μL of 80 mM glucose, 9 μM oligomycin, and 1M 2-deoxyglucose (for ECAR measurement) or 10 μM oligomycin, 9 μM FCCP, 10 μM rotenone, 10 μM antimycin A (for OCR measurement), was loaded in XF assay media into the injection ports in the XFe96 sensor cartridge. Measurements were normalized by protein content (SRB assay) and Hoechst 33342 content. Data sets were analyzed using XFe96 software and GraphPad Prism software, using one-way ANOVA and Student's t-test calculations. All experiments were performed in quintuplicate, three times independently.

Cell Cycle Analysis by FACS: Cell-cycle analysis was performed on the ATP-high and ATP-low cell sub-populations, by FACS analysis using the Attune NxT Flow Cytometer. Briefly, after trypsinization, the re-suspended cells were incubated with 10 ng/ml of Hoescht solution (Thermo Fisher Scientific) for 40 min at 37° C. under dark conditions. Following a 40 minute period, the cells were washed and re-suspended in PBS Ca/Mg for acquisition or in sorting buffer [1× PBS containing 3% (v/v) FBS and 2 mM EDTA] for FACS. 50,000 events were analyzed per condition. Gated cells were manually-categorized into cell-cycle stages.

Statistical Significance: All analyses were performed with GraphPad Prism 6. Data were represented as mean±SD (or ±SEM where indicated). All experiments were conducted at least 3 times independently, with >3 technical replicates for each experimental condition tested (unless stated otherwise, e.g., when representative data is shown). Statistically significant differences were determined using the Student's t-test or the analysis of variance (ANOVA) test. For the comparison among multiple groups, one-way ANOVA was used to determine statistical significance. p<0.05 was considered significant and all statistical tests were two-sided: p* <0.05; p** <0.01; p*** <0.005; p**** <0.0001.

Bioinformatic analysis: Unbiased label-free proteomics, comparing 2D-monolayers and 3D-mammospheres, was carried out as previously described, using MCF7 and T47D breast cancer cell lines. Informatics analysis was performed using a variety of publicly available of GEO DataSets (GSE36953; GSE2034; GSE59000; GSE55470), archived in the NCBI database, related to 3D growth, metastasis and circulating tumor cells (CTCs). Gene expression profiling data was extracted from these GEO DataSets. HeatMaps were generated with QIAGEN OmicSoft Suite Software. Volcano plots were produced by examining the annotations present in OncoLand Metastatic Cancer (QIAGEN OmicSoft Suite). In addition, functional “core analyses” was performed using Ingenuity Pathway Analysis Software (IPA; QIAGEN), on annotated genes. Gene co-expression profiles were extracted from The Metastatic Breast Cancer Project Provisional (2020), using cBioPortal (https://www.cbioportal.org/); mRNA expression profiling (RNA Seq V2 RSEM) was carried via RNA-sequencing of metastatic breast cancer samples from 146 patients.

Kaplan-Meier (K-M) analysis: To perform K-M analysis on ATP5F1C, we used an open-access online survival analysis tool to interrogate publicly-available microarray data from up to 3,951 breast cancer patients. For this purpose, we primarily analyzed data from ER(+) patients. Biased array data were excluded from the analysis. This allowed us to identify ATP5F1C (also known as ATP5C1), as a significant prognostic marker. Hazard-ratios were calculated, at the best auto-selected cut-off, and p-values were calculated using the Log-rank test and plotted in R. K-M curves were generated online using the K-M-plotter (as high-resolution TIFF files), using univariate analysis:

https ://kmplot.com/analysis/index.php?p=service&cancer=breast.

This approach allowed for directly performing in silico validation of ATP5F1C as a marker of tumor recurrence (RFS, replapse-free survival) and distant metastasis (DMFS, distant metastasis-free survival). The latest 2020 version of the database was utilized for all these analyses.

Cell Migration Assays: Briefly, 2.5×104 cells in 0.5 ml of serum-free DMEM with 0.1% BSA were added to the wells of 8-μm pore, non-coated membrane modified Boyden chambers (Transwells). The lower chambers contained 10% fetal bovine serum in DMEM to serve as a chemo-attractant. Cells were incubated at 37° C. and allowed to migrate throughout the course of 6 h. Noninvasive cells were removed from the upper surface of the membrane by scrubbing with cotton swabs. Chambers were stained in 0.5% crystal violet diluted in 100% methanol for 30-60 min, rinsed in water and examined under a bright-field microscope. Values for invasion and migration were obtained by counting five fields per membrane (20x objective) and represent the average of three independent experiments. Note that Transwells, pre-coated with extracellular matrix (namely Matrigel), were used to measure aggressive cell invasion and prevent simple cell migration.

Metastasis Assays: The chick embryo metastasis assay was performed by INOVOTION (Societe: 811310127), La Tronche-France. According to the French legislation, no ethical approval is needed for scientific experimentations using oviparous embryos (decree n° 2013-118, Feb. 1, 2013; art. R-214-88). Animal studies were performed under animal experimentation permit N° 381029 and B3851610001 to INOVOTION. Fertilized White Leghorn eggs were incubated at 37.5° C. with 50% relative humidity for 9 days. Greater than 20 eggs were processed for each experimental condition. At that moment (E9), the chorioallantoic membrane (CAM) was dropped down by drilling a small hole through the eggshell into the air sac, and a 1 cm² window was cut in the eggshell above the CAM. The MDA-MB-231 tumor cell line was cultivated in DMEM medium supplemented with 10% FBS and 1% penicillin/streptomycin. On day E9, cells were detached with trypsin, washed with complete medium and suspended in graft medium. After ATP-based cell sorting by flow-cytometry, an inoculum of 30,000 cells was added onto the CAM of each egg (E9) and then eggs were randomized into groups. On day E18, a 1 cm² portion of the lower CAM was collected to evaluate the number of metastatic cells in 8 samples per group (n=10). Genomic DNA was extracted from the CAM (commercial kit) and analyzed by qPCR with specific primers for Human Alu sequences. Calculation of Cq for each sample, mean Cq and relative amounts of metastases for each group are directly managed by the Bio-Rad® CFX Maestro software. Non-injected eggs were also evaluated in parallel, as a negative control for specificity. A one-way ANOVA analysis with post-tests was performed on all the data.

The terminology used in the description of embodiments of the present approach is for the purpose of describing particular embodiments only and is not intended to be limiting. As used in the description and the appended claims, the singular forms “a,” “an” and “the” are intended to include the plural forms as well, unless the context clearly indicates otherwise. The present approach encompasses numerous alternatives, modifications, and equivalents as will become apparent from consideration of the following detailed description.

It will be understood that although the terms “first,” “second,” “third,” “a),” “b),” and “c),” etc. may be used herein to describe various elements of the present approach, and the claims should not be limited by these terms. These terms are only used to distinguish one element of the present approach from another. Thus, a first element discussed below could be termed an element aspect, and similarly, a third without departing from the teachings of the present approach. Thus, the terms “first,” “second,” “third,” “a),” “b),” and “c),” etc. are not intended to necessarily convey a sequence or other hierarchy to the associated elements but are used for identification purposes only. The sequence of operations (or steps) is not limited to the order presented in the claims.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. All publications, patent applications, patents and other references mentioned herein are incorporated by reference in their entirety. In case of a conflict in terminology, the present specification is controlling.

Also, as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the present approach described herein can be used in any combination. Moreover, the present approach also contemplates that in some embodiments, any feature or combination of features described with respect to demonstrative embodiments can be excluded or omitted.

As used herein, the transitional phrase “consisting essentially of” (and grammatical variants) is to be interpreted as encompassing the recited materials or steps “and those that do not materially affect the basic and novel characteristic(s)” of the claim. Thus, the term “consisting essentially of” as used herein should not be interpreted as equivalent to “comprising.”

The term “about,” as used herein when referring to a measurable value, such as, for example, an amount or concentration and the like, is meant to encompass variations of ±20%, ±10%, ±5%, ±1%, ±0.5%, or even ±0.1% of the specified amount. A range provided herein for a measurable value may include any other range and/or individual value therein.

Having thus described certain embodiments of the present approach, it is to be understood that the scope of the appended claims is not to be limited by particular details set forth in the above description as many apparent variations thereof are possible without departing from the spirit or scope thereof as hereinafter claimed.

What is claimed is:

1. A purified composition of hyper-proliferative cancer stem cells comprising a sub-population of cells in a human cancer cell population, the cancer cell population expressing a range of fluorescent signals in response to a fluorescent adenosine triphosphate (ATP) imaging probe, and the sub-population of cells expressing an upper portion of the range of ATP-based fluorescent signals.

2. The composition of claim 1, wherein the upper portion comprises the top 10% of ATP-based fluorescent signals.

3. The composition of claim 1, wherein the upper portion comprises the top 5% of ATP-based fluorescent signals.

4. The composition of claim 1, wherein the composition is positive for one of a CD44 marker and an ALDH marker.

5. The composition of claim 1, wherein the composition comprises circulating tumor cells (CTC).

6. The composition of claim 1, wherein the composition is frozen.

7. A purified cell composition comprising a cancer stem cell sub-population stained with a fluorescent adenosine triphosphate (ATP) imaging probe and expressing a target portion of an ATP-based fluorescent signal range of a cancer cell population.

8. The composition of claim 7, wherein the cancer cell population expresses a range of ATP-based fluorescent signals, and the target portion of the ATP-based fluorescent signal range is one of an upper portion of the ATP-based fluorescent signals and a lower portion of the ATP-based fluorescent signals.

9. The composition of claim 8, wherein the target portion is one of the top 10% of ATP-based fluorescent signals and the top 5% of ATP-based fluorescent signals.

10. The composition of claim 8, wherein the target portion is one of the bottom 10% of ATP-based fluorescent signals and the bottom 5% of ATP-based fluorescent signals.

11. The composition of claim 9, wherein the composition is positive for one of a CD44 marker and an ALDH marker.

12. The composition of claim 1, wherein the sub-population of cells is stained with a fluorescent ATP imaging dye.

13. A purified composition of cells obtained by staining a human cancer cell population with a fluorescent adenosine triphosphate (ATP) imaging probe, separating a fraction of the human cancer cell population having a target portion of ATP-based fluorescent signals, and purifying the separated cells.

14. The composition of claim 13, wherein the target portion comprises one of the top 10% of ATP-based fluorescent signals, the top 5% of ATP-based fluorescent signals, the bottom 10% of ATP-based fluorescent signals, and the bottom 5% of ATP-based fluorescent signals.

15. The composition of claim 13, wherein the target portion comprises one of the top 10% of ATP-based fluorescent signals, the top 5% of ATP-based fluorescent signals, and the separated cells are positive for one of a CD44 marker and an ALDH marker.

16. The composition of claim 13, wherein the fluorescent imaging probe comprises ATP-Red-1.

17. A method of ATP-based cell fractionation, the method comprising:
staining cells in a cell population with a fluorescent adenosine triphosphate (ATP) imaging probe that fluoresces when bound to ATP;
measuring the ATP-based fluorescent signals of the stained cells in the cell population; and
separating the stained cells based on a target portion of ATP-based fluorescent signals.

18. The method of claim 17, wherein the target portion comprises one of the top 10% of ATP-based fluorescent signals, the top 5% of ATP-based fluorescent signals, the bottom 10% of ATP-based fluorescent signals, and the bottom 5% of ATP-based fluorescent signals.

19. The method of claim 17, wherein separating the stained cells based on target portion of ATP-based fluorescent signals comprises fluorescence-activated cell sorting (FACS) gating of the target portion of ATP-based fluorescent signals.

20. The method of claim 19, wherein the gates are set to collect at least one of (i) the stained cells having the top 10% of measured ATP-based fluorescent signals, and (ii) the stained cells having the bottom 10% of measured ATP-based fluorescent signals.

21. The method of claim 17, wherein the fluorescent ATP imaging probe comprises ATP-Red 1.

22. The method of claim 17, wherein the cell population is derived from one of blood, urine, saliva, tumor tissue, non-cancerous tissue, and a metastatic lesion.

23. The method of claim 17, further comprising at least one of measuring ALDH activity of separated cells, measuring anchorage-independent growth of separated cells, measuring the mitochondrial mass of separated cells, measuring the glycolytic and oxidative mitochondrial metabolism of separated cells, measuring the cell cycle progression and proliferative rate of separated cells, and measuring the poly-ploidy of separated cells.

24. A method for separating and collecting metabolically hyper-proliferative cells from a cell population, the method comprising:
staining cells in a cell population with an ATP-labeling dye, wherein the ATP-labeling dye fluoresces when bound to ATP;
measuring the ATP-based fluorescent signals of the stained cells in the cell population;
separating the stained cells based on the measured ATP-based fluorescent signals; and
collecting at least a portion of the separated cells having a measured ATP-based fluorescent signal one of above a predetermined threshold and below a predetermined threshold.

25. The separating and collecting method of claim 24, wherein the ATP-labeling dye comprises ATP-Red 1.

26. The separating and collecting method of claim 24, wherein the predetermined threshold comprises a percentage of an upper portion of the measured ATP-based fluorescent signals.

27. The separating and collecting method of claim 26, wherein the predetermined threshold comprises one of the top 25%, the top 20%, the top 15%, the top 10%, the top 5%, the top 2%, and the top 1%.

28. The separating and collecting method of claim 24, wherein separating and collecting is performed using fluorescence-activated cell sorting (FACS).

29. The separating and collecting method of claim 24, wherein the separated cells are further separated based on a second marker.

30. The separating and collecting method of claim 29, wherein the second marker comprises one of CD44(+), CD133(+), ESA(+), ALDEFLOUR(+), MitoTracker-High, EpCAM(+), CD90(+), CD34(+), CD29(+), CD73(+), CD90(+), CD105(+), CD106(+), CD166(+), and Stro-1(+).

31. The separating and collecting method of claim 30, wherein separating cells based on a second marker occurs at least one of (i) prior to staining cells in the cell population with the ATP-labeling dye, and (ii) after staining cells in the cell population with the ATP-labeling dye.

32. The separating and collecting method of claim 31, wherein the second marker comprises an antibody coated on magnetic beads.

33. The separating and collecting method of claim 24, further comprising staining the cells in the cell population with a second marker, and wherein the measuring the ATP-based fluorescent signals of the stained cells in the cell population occurs after staining with the second marker and the ATP-labeling dye.

34. A method for identifying and treating cancer stem cells in a biologic sample, the method comprising:
obtaining a biologic sample from a patient;
staining cells in the biologic sample with an ATP-labeling dye, wherein the ATP-labeling dye fluoresces when bound to ATP;
measuring the ATP-based fluorescent signals of the stained cells in the cell population;
comparing the measured ATP-based fluorescent signals to a predetermined threshold indicating the presence of cancer stem cells; and
if the measured ATP-based fluorescent signals exceeds the predetermined threshold, administering to the patient at least one ATP-depletion therapeutic.

35. The method of claim 34, wherein the ATP-depletion therapeutic comprises one of Doxycycline, Tigecycline, Azithromycin, Pyrvinium pamoate, Atovaquone, Bedaquiline, Niclosamide, Irinotecan, Actinonin, CAPE, Berberine, Brutieridin, Melitidin, Oligomycin, AR-C155858, a Mitoriboscin, a Mitoketoscin, a Mitoflavoscin, a TPP-derivative, dodecyl-TPP, 2-Butene-1,4-bis-TPP, Doxycycline conjugated with a fatty acid, and a combination of Doxycycline, Azithromycin and Ascorbic acid.

36. A method of testing a candidate compound for anti-cancer activity, the method comprising:
staining a cancer cell population with an ATP-labeling dye, wherein the ATP-labeling dye fluoresces when bound to ATP;
measuring the ATP-based fluorescent signals of the stained cells;
separating the stained cells based on a target portion of ATP-based fluorescent signals to prepare a hyper-active cancer cell sub-population;
administering the candidate compound to the hyper-active cancer cell sub-population; and
measuring the effect of the candidate compound on the hyper-active cancer cell sub-population.

37. The method of claim 36, wherein the ATP-labeling dye comprises ATP-Red 1.

38. The method of claim 36, wherein the target portion of ATP-based fluorescent signals comprises one of the top 25%, the top 20%, the top 15%, the top 10%, the top 5%, the top 2%, and the top 1%.

39. The method of claim 36, wherein the hyper-active cancer cell sub-population is positive for one of a CD44 marker an ALDH marker.

40. The method of claim 36, further comprising at least one of measuring ALDH activity of the hyper-active cancer cell sub-population, measuring anchorage-independent growth of the hyper-active cancer cell sub-population cells, measuring the mitochondrial mass of the hyper-active cancer cell sub-population, measuring the glycolytic and oxidative mitochondrial metabolism of the hyper-active cancer cell sub-population, measuring the cell cycle progression and proliferative rate of the hyper-active cancer cell sub-population, and measuring the poly-ploidy of the hyper-active cancer cell sub-population.

41. A method of diagnosing and preventing a risk of metastasis in a cancer patient, comprising:
determining the expression levels of ABCA2, ATP5F1C, COX20, NDUFA2, and UQCRB, in a biologic sample of the patient's cancer;
comparing the detected expression levels to baseline expression levels of ABCA2, ATP5F1C, COX20, NDUFA2, and UQCRB, in a non-cancerous biologic sample from the patient; and
if the detected expression levels exceed the baseline expression levels, administering an ATP-depletion compound to the patient.

42. The method of claim 41, wherein the ATP-depletion compound comprises one of Doxycycline, Tigecycline, Azithromycin, Pyrvinium pamoate, Atovaquone, Bedaquiline, Niclosamide, Irinotecan, Actinonin, CAPE, Berberine, Brutieridin, Melitidin, Oligomycin, AR-C155858, a Mitoriboscin, a Mitoketoscin, a Mitoflavoscin, a TPP-derivative, dodecyl-TPP, 2-Butene-1,4-bis-TPP, Doxycycline conjugated with a fatty acid, and a combination of Doxycycline, Azithromycin and Ascorbic acid.

43. A kit for identifying circulating tumor cells (CTCs) in a biologic sample, the kit comprising reagents for identifying an up-regulation of ABCA2, ATP5F1C, COX20, NDUFA2, and UQCRB in the biologic sample.

44. The kit of claim 43, wherein the reagents comprise at least one antibody directed at one of ABCA2, ATP5F1C, COX20, NDUFA2, and UQCRB.

45. A method for detecting circulating tumor cells (CTCs) in a biologic sample, the method comprising:
determining the expression levels of ABCA2, ATP5F1C, COX20, NDUFA2, and UQCRB, in the biologic sample; and
indicating the presence of CTCs if the determined expression levels are upregulated relative to a control.

46. The method of claim 45, wherein the biologic sample comprises one of blood, urine, saliva, tumor tissue, non-cancerous tissue, and a metastatic lesion.

47. The method of claim 45, further comprising separating CTCs from the biologic sample by staining the sample with a fluorescent ATP-labeling dye, measuring the ATP-based fluorescent signals of the stained sample; separating the stained sample based on a target portion of ATP-based fluorescent signals; and collecting the cell sub-population having the target portion of AATP-based fluorescent signals.

48. The method of claim 47, wherein the target portion of ATP-based fluorescent signals comprises one of the top 25%, the top 20%, the top 15%, the top 10%, the top 5%, the top 2%, and the top 1%.
